(19) United States
(12) Patent Application Publication
(10) Pub. No.: US 2023/0416346 A1
(43) Pub. Date: Dec. 28, 2023

(54) C-TERMINAL SPARC FRAGMENTS FOR TREATING CANCER

(71) Applicants: INSERM (INSTITUT NATIONAL DE LA SANTÉ ET DE LA RECHERCHE MÉDICALE), Paris (FR); INSTITUT RÉGIONAL DU CANCER DE MONTPELLIER, Montpellier (FR); UNIVERSITÉ DE MONTPELLIER, Montpellier (FR)

(72) Inventors: Emmanuelle LIAUDET-COOPMAN, Montpellier (FR); Lindsay ALCARAZ CACCHIA, Montpellier (FR); Aude MALLAVIALLE, Montpellier (FR)

(21) Appl. No.: 18/250,018

(22) PCT Filed: Oct. 20, 2021
(86) PCT No.: PCT/EP2021/079108

§ 371 Date: Apr. 21, 2023

(30) Foreign Application Priority Data
Oct. 21, 2020 (EP) 20306254.2

(51) Int. Cl. C07K 16/18; A61P 35/00; A61K 45/06

(52) U.S. Cl. CPC C07K 16/18; A61P 35/00; A61K 45/06

(57) ABSTRACT

Tumour-specific molecular targets and alternative therapeutic strategies for triple-negative breast cancer (TNBC) are urgently needed. The protease cathepsin D (cath-D) is aberrantly secreted and a marker of poor prognosis in breast cancer. Using degradomic analyses by TAILS, we discovered that the matricellular protein SPARC is a substrate of extracellular cath-D. In vitro, cath-D induced limited proteolysis of SPARC C-terminal extracellular Ca2+ binding domain at acidic pH, leading to the production of SPARC fragments (34-, 27-, 16-, 9-, and 6-kDa). SPARC cleavage also occurred in vivo in TNBC and mouse mammal tumours. Moreover, the C-terminal 9-kDa SPARC fragment inhibited MDA-MB-231 TNBC cell adhesion and spreading on fibronectin, and stimulated their migration, endothelial transmigration and invasion more potently than full-length SPARC. These results highlight a novel crosstalk between proteases and matricellular proteins in the TNBC microenvironment through limited proteolysis of SPARC, and reveal that the 9-kDa C-terminal SPARC fragment is an attractive therapeutic target for TNBC. Thus, the invention relates to an inhibitor of SPARC fragment for use for treating cancer, and in particularly triple cancer negative breast cancer.

### FIELD OF THE INVENTION

The present invention relates to cancer field. More particularly, the invention relates to use of an inhibitor of SPARC fragment in the treatment of cancer, particularly of triple negative breast cancer.

### BACKGROUND OF THE INVENTION

Breast cancer (BC) is one of the leading causes of death in women in developed countries. Triple-negative breast cancer (TNBC), defined by the absence of oestrogen receptor (ER), progesterone receptor (PR) and human epidermal growth factor receptor 2 (HER-2) overexpression and/or amplification, accounts for 15-20% of all BC cases¹. Chemotherapy is the primary systemic treatment, but resistance to this treatment is common¹. Thus, tumour-specific molecular targets are urgently needed to develop alternative therapeutic strategies for TNBC.

Human cathepsin D (cath-D) is a ubiquitous, lysosomal, aspartic endoproteinase that is proteolytically active at acidic pH. Cath-D expression levels in breast cancer (BC)²⁻⁴ and triple negative breast cancer (TNBC)^{5,6} correlate with poor prognosis. Cath-D over-production by BC and TNBC cells leads to hypersecretion of the 52-kDa cath-D precursor in the extracellular environment^{6,7}. Purified 52-kDa cath-D self-activates in acidic conditions, giving rise to a catalytically active 51-kDa pseudo-cath-D form that retains the 18 residues (27-44) of the pro-segment⁸. Cath-D affects the tumour and its microenvironment by increasing proliferation of BC cells^{7,9-11}, and by stimulating mammary fibroblast outgrowth^{12,13} angiogenesis^{9,14}, and metastasis formation¹¹. However, little is known about the molecular mechanisms and the substrates involved in these processes. The inventors previously indicated that cath-D is a tumour-specific extracellular target in TNBC and its suitability for antibody-based therapy¹⁵. Thus, the identification of the cath-D extracellular substrate repertoire by N-Terminal Amine Isotopic Labeling of Substrates (N-TAILS) degradomics¹⁶ is important for the mechanistic understanding of its role in TNBC. In a previous work using TAILS¹⁷, the inventors isolated the matricellular protein SPARC (Secreted Protein Acidic and Rich in Cysteine), also known as osteonectin or basement membrane 40 (BM40), as a putative cath-D substrate.

SPARC is a Ca2+-binding glycoprotein that regulates extracellular matrix assembly and deposition, growth factor signalling, and interactions between cells and their surrounding extracellular matrix¹⁸⁻²¹. In cancer, SPARC is mainly secreted by the neighbouring stroma, but also by cancer cells²²⁻²⁴. In different cancer types, SPARC plays an oncogenic or a tumour-suppressive role²⁵⁻²⁶. For instance, in breast cancer, SPARC has a pro-tumorigenic role and has been associated with worse prognosis²⁷⁻³³; however, other studies reported anti-tumorigenic functions³⁴⁻³⁶. SPARC includes three different structural and functional modules: the N-terminal acidic domain, followed by the follistatin-like domain, and the C-terminal extracellular Ca2+ binding domain¹⁸. Protein fragments that correspond to these SPARC domains display distinct biological functions in cell de-adhesion and spreading, motility, proliferation, invasion, and in matrix remodelling³⁷⁻³⁹.

Herein, the inventors found that in the acidic tumour microenvironment of triple negative breast cancer, cath-D cleaved SPARC exclusively in its C-terminal extracellular Ca2+ binding domain releasing five main fragments (34-, 27-, 16-, 9-, and 6-kDa). Among these fragments, the 9-kDa C-terminal SPARC fragment (amino acids 235-303) had greater oncogenic activity than FL SPARC, highlighting the importance of limited proteolysis of matricellular proteins in the TNBC microenvironment. This knowledge might pave the way to the development of strategies to target the bioactive fragments of matricellular proteins in cancer.

### SUMMARY OF THE INVENTION

The inventor demonstrated that the matricellular protein SPARC is an extracellular cath-D substrate. They showed that cath-D secreted by TNBC cells cleaves fibroblast- and cancer-derived SPARC at the pericellular pH of the tumour, leading to the production of SPARC fragments (34-, 27-, 16-, 9- and 6-kDa). SPARC cleavage also occurred in TNBC tumours. Among these fragments, the C-terminal 9-kDa SPARC fragment inhibited TNBC cell adhesion and spreading, and stimulated their migration, endothelial transmigration, and invasion more potently than full-length SPARC.

Thus the present invention relates to an inhibitor of SPARC fragment for use for treating cancer in a subject in need thereof. More particularly, the invention is defined by its claims.

### DETAILED DESCRIPTION OF THE INVENTION

Herein, the inventors found that in the acidic tumour microenvironment of triple negative breast cancer, cath-D cleaved SPARC exclusively in its C-terminal extracellular Ca2+ binding domain releasing five main fragments (34-, 27-, 16-, 9-, and 6-kDa). Among these fragments, the 9-kDa C-terminal SPARC fragment (amino acids 235-303) had greater oncogenic activity than FL SPARC, highlighting the importance of limited proteolysis of matricellular proteins in the TNBC microenvironment. This knowledge pave the way to the development of strategies to target the bioactive fragments of matricellular proteins in cancer.

### Therapeutic Methods and Uses

Accordingly, in a first aspect the present invention relates to a method for treating cancer in a subject in need thereof comprising administering an effective amount of an inhibitor of SPARC fragment.

In other word, the invention relates to an inhibitor of SPARC fragment for use for treating cancer in a subject in need thereof.

As used herein, the term “SPARC”, for “Secreted Protein Acidic and Rich in Cysteine”, has its general meaning in the art and refers to a Ca2+-binding glycoprotein that regulates extracellular matrix assembly and deposition, growth factor signalling, and interactions between cells and their surrounding extracellular matrix. In cancer, SPARC is mainly secreted by the neighbouring stroma, but also by cancer cells²²⁻²⁴ and plays an oncogenic or a tumour-suppressive role²⁵⁻²⁶.

Inventors demonstrate that cath-D induced limited proteolysis of SPARC C-terminal extracellular Ca²⁺ binding domain at acidic pH, leading to the production of SPARC fragments of different weight (34-, 27-, 16-, 9-, and 6-kDa).

As used herein, the term “Cath-D” has its general meaning in the art and refers to lysosomal aspartic protease cathepsin-D. Cath-D is synthesized as the 52 kDa, catalytically inactive, precursor called pro-Cath-D. It is present in endosomes as an active 48 kDa single-chain intermediate that is subsequently converted in the lysosomes into the fully active mature protease, composed of a 34 kDa heavy and a 14 kDa light chains. The naturally occurring pro-cath-D protein has an amino acid sequence shown in Genbank, Accession number NP_001900.

Accordingly to the invention, the term SPARC fragment refers to peptides produced by the proteolysis of SPARC C-terminal extracellular Ca²⁺ binding domain by Cath-D. The term SPARC fragment include C-terminal 34-, 27-, 16-, 9- and 6-kDa SPARC fragment.

In some embodiment, the SPARC fragment comprises or consists of the peptides in table 1.

TABLE 1
Sequences of 34-, 27-, 16-, 9- and 6-kDa SPARC fragments.

| | SEQ | |
|---|---|---|
| | ID | |
| SPARC Fragments | NO: | SPARC peptides |
| Fragments 34-kDa | 1 | MRAWIFFLLCLAGRALAAPQQEALPDETEVVEETVAEV |
| 1-257 | | TEVSVGANPVQVEVGEFDDGAEETEEEVVAENPCQNH |
| | | HCKHGKVCELDENNTPMCVCQDPTSCPAPIGEFEKVCS |
| | | NDNKTFDSSCHFFATKCTLEGTKKGHKLHLDYIGPCKYI |
| | | PPCLDSELTEFPLRMRDWLKNVLVTLYERDEDNNLLTE |
| | | KQKLRVKKIHENEKRLEAGDHPVELLARDFEKNYNMYI |
| | | FPVHWQFGQLDQHPIDGYLSHTELAPLRA |
| Fragments 27-kDa | 2 | MRAWIFFLLCLAGRALAAPQQEALPDETEVVEETVAEV |
| 1-206 | | TEVSVGANPVQVEVGEFDDGAEETEEEVVAENPCQNH |
| | | HCKHGKVCELDENNTPMCVCQDPTSCPAPIGEFEKVCS |
| | | NDNKTFDSSCHFFATKCTLEGTKKGHKLHLDYIGPCKYI |
| | | PPCLDSELTEFPLRMRDWLKNVLVTLYERDEDNNLLTE |
| | | KQKLRVKKIHENEKRL |
| Fragments 16-kDa | 3 | VTLYERDEDNNLLTEKQKLRVKKIHENEKRLEAGDHPV |
| 176-303 | | ELLARDFEKNYNMYIFPVHWQFGQLDQHPIDGYLSHTE |
| | | LAPLRAPLIPMEHCTTRFFETCDLDNDKYIALDEWAGCF |
| | | GIKQKDIDKDLVI |
| Fragments 16-kDa | 4 | YERDEDNNLLTEKQKLRVKKIHENEKRLEAGDHPVELL |
| 179-303 | | ARDFEKNYNMYIFPVHWQFGQLDQHPIDGYLSHTELAP |
| | | LRAPLIPMEHCTTRFFETCDLDNDKYIALDEWAGCFGIK |
| | | QKDIDKDLVI |
| Fragments 9-kDa | 5 | PVHWQFGQLDQHPIDGYLSHTELAPLRAPLIPMEHCTTR |
| 230-303 | | FFETCDLDNDKYIALDEWAGCFGIKQKDIDKDLVI |
| Fragments 9-kDa | 6 | WQFGQLDQHPIDGYLSHTELAPLRAPLIPMEHCTTRFFE |
| 233-303 | | TCDLDNDKYIALDEWAGCFGIKQKDIDKDLVI |
| Fragments 9-kDa | 7 | FGQLDQHPIDGYLSHTELAPLRAPLIPMEHCTTRFFETC |
| 235-303 | | DLDNDKYIALDEWAGCFGIKQKDIDKDLVI |
| Fragments 9-kDa | 8 | GQLDQHPIDGYLSHTELAPLRAPLIPMEHCTTRFFETCD |
| 236-303 | | LDNDKYIALDEWAGCFGIKQKDIDKDLVI |
| Fragments 9-kDa | 9 | QLDQHPIDGYLSHTELAPLRAPLIPMEHCTTRFFETCDL |
| 237-303 | | DNDKYIALDEWAGCFGIKQKDIDKDLVI |
| Fragments 9-kDa | 10 | DQHPIDGYLSHTELAPLRAPLIPMEHCTTRFFETCDLDN |
| 239-303 | | DKYIALDEWAGCFGIKQKDIDKDLVI |
| Fragments 9-kDa | 11 | YLSHTELAPLRAPLIPMEHCTTRFFETCDLDNDKYIALD |
| 246-303 | | EWAGCFGIKQKDIDKDLVI |
| Fragments 9-kDa | 12 | LSHTELAPLRAPLIPMEHCTTRFFETCDLDNDKYIALDE |
| 247-303 | | WAGCFGIKQKDIDKDLVI |
| Fragments 9-kDa | 13 | SHTELAPLRAPLIPMEHCTTRFFETCDLDNDKYIALDEW |
| 248-303 | | AGCFGIKQKDIDKDLVI |
| Fragments 6-kDa | 14 | PLIPMEHCTTRFFETCDLDNDKYIALDEWAGCFGIKQKD |
| 258-303 | | IDKDLVI |

In some embodiment, the SPARC fragment comprises or consists of the peptides selected from the group consisting in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, and SEQ ID NO:14.

Thus, the present invention relates a method for treating cancer in a subject in need thereof comprising administering an effective amount of an inhibitor of SPARC fragment, wherein the SPARC fragment comprises or consist of the peptides selected from the group consisting in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, and SEQ ID NO:14.

The inventors demonstrate that among the fragments of SPARC, the C-terminal 9-kDa SPARC fragment inhibited TNBC cell adhesion and spreading, and stimulated their migration, endothelial transmigration, and invasion more potently than full-length SPARC.

Accordingly, in some embodiment the SPARC fragment is a C-terminal 9-kDa SPARC fragment.

In some embodiment, the C-terminal 9-kDa SPARC fragment is a peptide selected from the group consisting in SEQ ID NO:5, SEQ ID NO:6, SEQ ID NO:7, SEQ ID NO:8, SEQ ID NO:9, SEQ ID NO:10, SEQ ID NO:11, SEQ ID NO:12, and SEQ ID NO:13.

Thus, the present invention relates a method for treating cancer in a subject in need thereof comprising administering an effective amount of an inhibitor of SPARC fragment, wherein the SPARC fragment consist of the peptides selected from the group consisting in SEQ ID NO:5, SEQ ID NO:6, SEQ ID NO:7, SEQ ID NO:8, SEQ ID NO:9, SEQ ID NO:10, SEQ ID NO:11, SEQ ID NO:12, and SEQ ID NO:13.

As used herein, an “inhibitor of SPARC fragment” refers to a natural or synthetic compound able to inhibit the activity of the SPARC fragment and selectively blocks or inactivates SPARC fragment. As used herein, the term “selectively blocks or inactivates” refers to a compound that preferentially binds to and blocks or inactivates SPARC fragment with a greater affinity and potency, respectively, than its interaction with Cath-D.

The “inhibitor of SPARC fragment” refers to compounds that block the cleavage of SPARC by Cath-D producing said SPARC fragment. In other word, the “inhibitor of SPARC fragment” includes also compound that block the proteolysis of SPARC by Cath-D, and in particular the proteolysis of the C-terminal extracellular Ca²⁺ binding domain, and more particularly the proteolysis of an C-terminal 9-kDa SPARC fragment. The “inhibitor of SPARC fragment” refers to compounds that block the oncogenic action of the SPARC fragment and in particular to a 9-kDa SPARC fragment.

In some embodiment, the inhibitor of SPARC fragment inhibits the migration, endothelial transmigration and invasion of cancer cells.

In some embodiment, the inhibitor of SPARC fragment for use according to the invention is an antibody, a peptide, a polypeptide, a small molecule or an aptamer.

Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against SPARC fragment of the invention as above described, the skilled man in the art can easily select those inhibiting SPARC fragment.

In some embodiment, the the inhibitor of SPARC fragment for use according to the invention is an antibody (the term including “antibody portion”).

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of SPARC fragment. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the antigen may be provided as synthetic peptides corresponding to antigenic regions of interest in SPARC fragment. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4. In a preferred embodiment, the inhibitor of SPARC fragment of the invention is a Human IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In some embodiment, the the inhibitor of SPARC fragment for use according to the invention is a polypeptide.

In particular embodiment, the polypeptide is an antagonist of SPARC fragment and is capable to prevent the function of SPARC fragment.

In one embodiment, the polypeptide of the invention may be linked to a cell-penetrating peptide” to allow the penetration of the polypeptide in the cell.

The term “cell-penetrating peptides” are well known in the art and refers to cell permeable sequence or membranous penetrating sequence such as penetratin, TAT mitochondrial penetrating sequence and compounds (Bechara and Sagan, 2013; Jones and Sayers, 2012; Khafagy el and Morishita, 2012; Malhi and Murthy, 2012).

The polypeptides of the invention may be produced by any suitable means, as will be apparent to those of skill in the art. In order to produce sufficient amounts of polypeptide or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known. When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli. In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters. A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications. Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa). In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

As used herein, the term “cancer” refers to a group of diseases involving abnormal cell growth with the potential to invade or spread to other parts of the body. The cancer that may treated by methods and compositions of the invention include, but are not limited to cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In a particular embodiment, the cancer includes, but is not limited to breast cancer, melanoma, ovarian cancer, lung cancer, liver cancer, pancreatic cancer, endometrial cancer, head and neck cancer, bladder cancer, malignant glioma, prostate cancer, colon adenocarcinoma or gastric cancer.

In some embodiment, the cancer is breast cancer.

As used herein, the term “breast cancer” has its general meaning in the art and refers to a cancer that forms in the cells of the breasts. Breast cancer include basal breast cancer, metastatic breast cancer or triple negative breast cancer. As used herein the expression “triple negative breast cancer” has its general meaning in the art and means that said breast cancer lacks receptors for the hormones estrogen (ER-negative) and progesterone (PR-negative), and for the protein HER2.

In some embodiment, the cancer is an estrogen-receptor positive (ER+) hormono-resistant breast cancer or a triple-negative (ER− and PR−, HER2-non amplified) breast cancer (TNBC).

In some embodiment, the cancer is a triple negative breast cancer.

As used herein, the term “subject” refers to any mammals, such as a rodent, a feline, a canine, and a primate. Particularly, in the present invention, the subject is a human afflicted with or susceptible to be afflicted with a disease wherein Cath-D is overexpressed. In another embodiment, the subject is a human afflicted with or susceptible to be afflicted with a cancer. In another embodiment, the subject is a human afflicted with or susceptible to be afflicted with TNBC.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the terms “administering” or “administration” refer to the act of injecting or otherwise physically delivering a substance as it exists outside the body (e.g., an anti-cath-D antibody) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

By a “therapeutically effective amount” is meant a sufficient amount of inhibitor of SPARC fragment of the invention for use in a method for the treatment of cancer at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The inhibitor of SPARC fragment for use according to the invention can be administered in combination with a classical treatment of cancer.

Thus, the invention also refers to i) an inhibitor of SPARC fragment and ii) a classical treatment of cancer for use for treating cancer in a subject in need thereof.

In other word, the invention refers to a method of treating cancer in a subject in need thereof, comprising administrating to said subject a therapeutically effective amount of an inhibitor of SPARC fragment and a classical treatment of cancer.

As used herein, the term “classical treatment” refers to any compound, natural or synthetic, used for the treatment of cancer.

In a particular embodiment, the classical treatment refers to radiation therapy, immunotherapy or chemotherapy.

According to the invention, compound used for the classical treatment of cancer may be selected in the group consisting in: EGFR inhibitor such as cetuximab, panitumumab, bevacizumab and ramucirumab; kinase inhibitor such as erlotinib, gefitinib afatinib, regorafenib and larotrectinib; immune checkpoint inhibitor; chemotherapeutic agent and radiotherapeutics agent.

As used herein, the term “chemotherapy” refers to cancer treatment that uses one or more chemotherapeutic agents. As used herein, the term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a camptothecin (including the synthetic analogue topotecan and irinotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, trifluridine, tipiracil, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum such as oxaliplatin, cisplatin and carbloplatin; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; ziv-aflibercept; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

As used herein, the term “radiation therapy” has its general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a colorectal cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, PD-L1, LAG-3, TIM-3 and VISTA.

According to the invention, the inhibitor of the SPARC fragment and the classical treatment can be used as a combined treatment.

As used herein, the terms “combined treatment”, “combined therapy” or “therapy combination” refer to a treatment that uses more than one medication. The combined therapy may be dual therapy or bi-therapy. The medications used in the combined treatment according to the invention are administered to the subject simultaneously, separately or sequentially.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

### Pharmaceutical Composition

The inhibitor of SPARC fragment for use of the invention may be used or prepared in a pharmaceutical composition.

In one embodiment, the invention relates to a pharmaceutical composition comprising the inhibitor of SPARC fragment for use in the treatment of cancer in a subject of need thereof.

In some embodiment, the cancer is breast cancer, and more particularly triple negative breast cancer.

Typically, the inhibitor of SPARC fragment may be combined with pharmaceutically acceptable excipients, and optionally sustained-release matrices, such as biodegradable polymers, to form therapeutic compositions.

As used herein, the term “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising inhibitors of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The inhibitor of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

TABLE 2
Sequences of cleaved SPARC fragments identified by ATOMS and TAILS

| | | | | ATOMS (in vitro cleavages | | | | | |
|---|---|---|---|---|---|---|---|---|---|
| | | | | of recombinant SPARC by | | | | | |
| | | | | recombinant cath-D) | | | | TAILS (SPARC | |
| | | | | (Pseudo-cathD + | | (Mature cath-D + | | cleavages from | |
| | | | | SPARC − | | SPARC − | | conditioned | |
| | | | | pepst./Pseudo- | | pepst./ | | media of | |
| | | | | cath-D + | | Mature cath- | | cocultures) | |
| | | | Enzyme | SPARC + | | D + SPARC + | | (CM − pepst./ | |
| | SEQ | | used for | pepst) ratio | | pepst.) ratio | | CM + pepst.)ratio | |
| | ID | SPARC | sample | (number of peptide | | (number of peptide | | (number of peptide | |
| SPARC peptides | NO: | fragments* | digestion | identifications) | | identifications) | | identifications) | |
| LDSELTEFPLR [156-166] | 15 | | Trypsin | | | | | 0.4 | (6) |
| DWLKNVLVTLYER [169-181] | 16 | | Trypsin | | | | | 0.5 | (3) |
| VTLYERDEDNNLLTEK [176-191] | 17 | 16-kDa | Trypsin | 12.1 | (5) | 14.3 | (5) | | |
| YERDEDNNLLTEK [179-191] | 18 | | Trypsin | 13.7 | (16) | 11.8 | (16) | | |
| YERDEDNNLLTEKQK [179-193] | 19 | | Trypsin | 21.1 | (15) | 16.8 | (15) | | |
| EAGDHPVELLAR [207-218] | 20 | 27-kDa | Trypsin | 11.3 | (6) | 19.3 | (6) | 3.4 | (2) |
| PVHWQFGQLDQHPIDGYLSHTELAPLR | 21 | 9-kDa | Trypsin | | | | | 5.6 | (8) |
| [230-256] | | | | | | | | | |
| WQFGQLDQHPIDGYLSHTELAPLR | 22 | | Trypsin | | | | | 3.1 | (3) |
| [233-256] | | | | | | | | | |
| FGQLDQHPIDGYLSHTELAPLR | 23 | | Trypsin | 10.6 | (5) | 5.9 | (5) | | |
| [235-256] | | | | | | | | | |
| GQLDQHPIDGYLSHTELAPLR | 24 | | Trypsin | 2 | (16) | 2.3 | (16) | 12.4 | (15) |
| [236-256] | | | | | | | | | |
| QLDQHPIDGYLSHTELAPLR | 25 | | Trypsin | 3.5 | (5) | 2.2 | (5) | 15.1 | (5) |
| [237-256] | | | | | | | | | |
| DQHPIDGYLSHTELAPLR [239-256] | 26 | | Trypsin | 8.4 | (18) | 8.1 | (18) | 7.6 | (18) |
| YLSHTELAPLR [246-256] | 27 | | Trypsin | 18.8 | (8) | 5.7 | (8) | 4.4 | (4) |
| LSHTELAPLR [247-256] | 28 | | Trypsin | | | | | 2.4 | (1) |
| SHTELAPLR [248-256] | 29 | | Trypsin | 2.7 | (5) | 1.6 | (5) | | |
| PLIPMEHCTTR [258-268] | 30 | 6- and | Trypsin | 22.5 | (15) | 4.3 | (15) | 5.9 | (3) |
| | | 34-kDa | | | | | | | |

| | | | | | |
|---|---|---|---|---|---|
| APQQEALPDE [18-27] | 31 | | Glu-C | 0.98 | (2) |
| APQQEALPDETE [18-29] | 32 | | Glu-C | 0.88 | (13) |
| APQQEALPDETEVVE [18-32] | 33 | | Glu-C | 1.12 | (6) |
| APQQEALPDETEVVEE [18-33] | 34 | | Glu-C | 1.1 | (140) |
| APQQEALPDETEVVEETVAE [18-37] | 35 | | Glu-C | 1.14 | (18) |
| VTLYERDEDNNLLTE [176-190] | 36 | 16.4-kDa | Glu-C | 14.3 | (12) |
| YERDEDNNLLTE [179-190] | 37 | | Glu-C | 13.1 | (43) |
| FGQLDQHPIDGYLSHTE [235-251] | 38 | 9-kDa | Glu-C | 9.2 | (3) |
| GOLDQHPIDGYLSHTE [236-251] | 39 | | Glu-C | 10.4 | (12) |
| DQHPIDGYLSHTE [239-251] | 40 | | Glu-C | 4.7 | (6) |

Ratio; <0.5 or >2 for trypin; Ratio; <0.5 or >2 in bold for Glu-C; CM, conditioned medium; pepst., pepstatin A; *, according the silver staining.

### FIGURES

FIG. 1. SPARC analysis in Ctsd−/− MEFs transfected with human cath-D. A. TAILS-based identification of protein N-termini affected by Ctsd deficiency. Comparison of the amounts of protein N termini in the secretomes of cath-D-deficient MEFs transfected with a plasmid encoding human cath-D (Ctsd−/−cath-D) or empty vector (Ctsd−/−). The distribution of 700 quantified N-terminal peptides was visualized as a raincloud plot that includes the distribution of individual N-terminal peptide ratios, a boxplot, and the probability distribution. Dashed lines indicate the 3-fold change in abundance (log2<−1.58 or >1.58) chosen as cut-off for N-termini considered as severely affected by cath-D expression. The arrow indicates the SPARC peptide LDSELTEFPLR [156-166] (SEQ ID NO:1). B. Effect of Ctsd deficiency on SPARC protein level. Secretomes (30 ∞g) of Ctsd−/− and Ctsd−/−cath-D MEFs were separated on 13.5% SDS-PAGE followed by immunoblotting with anti-cath-D (clone 49, #610801) and anti-SPARC (clone AON-5031) antibodies. C. Effect of Ctsd deficiency on Sparc mRNA level by transcriptomic analysis. The boxplots show the distribution of the expression values for all mRNAs (in log2 of ratio) in Ctsd−/−cath-D and Ctsd−/MEFs for two independent experiments. Arrows indicate Sparc expression values (0.91 and 0.87 on a linear scale, corresponding to −0.14 and −0.19 in the log2 scale). In these two experiments, Sparc mRNA showed a minimal deviation from the threshold (fold change of 2 or ½; i.e. log2(R)+1 or −1) commonly used to consider a gene as up- or down-regulated, and indicated by the dashed lines.

FIG. 2. Cleavage of human SPARC in its extracellular Ca2+ binding domain by human cath-D at acidic pH. A. Time-course of cath-D-induced SPARC cleavage. Recombinant human FL SPARC was incubated with auto-activated cath-D in cleavage buffer at pH 5.9 with or without pepstatin A (Pepst.) at 37° C. for the indicated times. SPARC cleavage was analysed by 13.5% SDS-PAGE and immunoblotting with an anti-SPARC monoclonal antibody (clone AON-5031). B. pH dependence of cath-D-induced SPARC cleavage. Recombinant human FL SPARC was incubated with auto-activated cath-D in cleavage buffer with or without pepstatin A (Pepst.) at the indicated pH at 37° C. overnight. SPARC cleavage was analysed as in (A). C. Detection of the cath-D-induced SPARC fragments by silver staining. Recombinant SPARC was incubated with recombinant auto-activated pseudo-cath-D (51-kDa) or fully-mature cath-D (34+14-kDa) at pH 5.9 for the indicated times. SPARC cleavage was analysed by 17% SDS-PAGE and silver staining. D. Cleavage sites by cath-D in SPARC extracellular Ca2+ binding domain. The entire C-terminal extracellular Ca2+ binding domain of human SPARC (amino acids 154-303) is shown. SPARC cleaved peptides generated in the extracellular Ca2+ binding domain by auto-activated pseudo-cath-D (51-kDa) and fully-mature (34+14-kDa) cath-D at pH 5.9 were resolved by iTRAQ-ATOMS. Arrows, cleavage sites. E. Schematic representation of the SPARC fragments generated by cath-D according to (C) and (D).

FIG. 3. Limited proteolysis of fibroblast- and cancer-derived SPARC by cath-D secreted by TNBC and mouse breast cancer cells at acidic pH. A. Time-course of SPARC degradation in the MDA-MB-231/HMF co-culture. MDA-MB-231 TNBC cells and HMFs were co-cultured in FCS-free DMEM without sodium bicarbonate and phenol red and buffered with 50 mM HEPES [pH 7] at 37° C. for 24 h. The 24 h conditioned medium from co-cultured MDA-MB-231/HMFs was incubated at 37° C. in cleavage buffer with or without pepstatin A (Pepst.) at pH 5.5 for the indicated times. SPARC cleavage in conditioned medium was analysed by 13.5% SDS-PAGE and immunoblotting with an anti-SPARC polyclonal antibody (Proteintech). O/N, overnight. B. Influence of the milieu acidity on SPARC degradation in the MDA-MB-231/HMF co-culture. MDA-MB-231 TNBC cells and HMFs were co-cultured as in (A). The 24-hour conditioned medium from co-cultured MDA-MB-231/HMFs was incubated at 37° C. in cleavage buffer with or without pepstatin A at the indicated pH overnight. SPARC cleavage was analysed as described in (A). C and D. Time-course of SPARC cleavage in conditioned medium. HS578T TNBC cells (C) and SUM159 TNBC cells (D) were cultured in FCS-free DMEM without sodium bicarbonate and phenol red and buffered with 50 mM HEPES [pH 7] at 37° C. for 24 h. The 24 h conditioned medium was incubated at 37° C. in cleavage buffer with or without pepstatin A at pH 5.5 for the indicated times. SPARC cleavage was analysed as described in (A). E. SPARC cleavage by cath-D secreted by MDA-MB-231 cells. MDA-MB-231 cells were transfected with Luc or cath-D siRNAs. At 48 h post-transfection, siRNA-transfected MDA-MB-231 cells were co-cultured with HMFs as described in (A). Then, the 24-hour conditioned media from co-cultured siRNA-transfected MDA-MB-231/HMFs were incubated at 37° C. in cleavage buffer with or without pepstatin A at pH 5.5 for 120 min. Cath-D secretion by siRNA-transfected MDA-MB-231 cells was analysed with an anti-cath-D antibody (H-75). SPARC cleavage was analysed as described in (A). F. SPARC cleavage by cath-D secreted by inducible Ctsd knock-out MMTV-PyMT mammary tumour cells. Inducible Ctsd knock-out MMTV-PyMT breast cancer cells were incubated or not with 4-hydroxytamoxifen (OH-Tam; 3 μM) for 4 days to induce Ctsd knock-out. Then, cells were cultured in FCS-free DMEM without sodium bicarbonate and phenol red and buffered with 50 mM HEPES [pH 7] at 37° C. for 24 h. This 24 h conditioned medium conditioned medium was incubated at 37° C. in cleavage buffer with or without pepstatin A at pH 5.5 for 120 min or O/N. Cath-D secretion was analysed with an anti-cath-D antibody (AF1029). SPARC cleavage was analysed as described in (A).

FIG. 4. Detection of FL SPARC and its cleaved fragments in mammary tumours. A. SPARC expression in mammary tumours from MMTV-PyMT Ctsd knock-out mice. Left panel, whole cytosols (40 μg) of mammary tumours from MMTV-PyMTCtsd+/+ (N° 1-3) and MMTV-PyMTCtsd−/− (Ctsd knock-down in the mammary gland) (N° 4-6) mice were analysed by 13.5% SDS-PAGE and immunoblotting with an anti-mouse cath-D monoclonal (clone 49, #610801) and anti-SPARC monoclonal antibody (AON-5031). β-actin, loading control. Right panel, total RNA was extracted from mammary tumours from MMTV-PyMTCtsd+/+ (N° 1-3) and MMTV-PyMTCtsd−/− (N° 4-6) mice. Sparc expression was analysed by RT-qPCR. P=0.1 (Student's t-test). B and C. SPARC expression in TNBC PDXs and TNBC biopsies. Top panels, cath-D expression was determined in whole cytosols from two TNBC PDXs (B) and two TNBC biopsies (C) by sandwich ELISA with the immobilized anti-human cath-D D7E3 antibody and the anti-human cath-D M1G8 antibody coupled to HRP. Bottom panels, whole cytosols (40 μg) from these PDXs (B) and TNBC biopsies (C) were analysed by 13.5% SDS-PAGE and immunoblotting with anti-cath-D (H-75) and anti-SPARC (Proteintech) polyclonal antibodies. β-actin (B) and tubulin (C), loading controls.

FIG. 5. Effects of FL SPARC and cath-D-induced cleaved SPARC fragments on adhesion, migration, transmigration and invasion of TNBC cells. A. Cell adhesion. MDA-MB-231 cells were let to adhere for 30 min on a fibronectin matrix in the absence or presence of recombinant FL SPARC (SPARC), or recombinant cath-D-induced cleaved SPARC fragments (cleaved SPARC) at the final concentration of 240 nM. Left panels, representative images of adherent cells. Right panel, adherent cells were stained with crystal violet, and adhesion was quantified at 570 nm. CTRL, PBS in cleavage buffer. Data are the mean±SD (n=3); ***, p<0.001, ANOVA and Bonferroni's post hoc test. Similar results were obtained in four independent experiments. B. Cell migration. MDA-MB-231 cells were let to migrate for 16 h on a fibronectin matrix in the absence or presence of recombinant FL SPARC, or cleaved SPARC at a final concentration of 240 nM. Left panels, representative images of migrating cells. Right panel, migrating cells were quantified by MTT staining and absorbance was read at 570 nm. CTRL, PBS in cleavage buffer. Data are the mean±SD (n=3); *, p<0.05; **, p<0.01; ***, p<0.001, ANOVA and Bonferroni's post hoc test. Similar results were obtained in three independent experiments. C. Endothelial transmigration. MDA-MB-231 cells were let to transmigrate for 16 h through a HUVEC monolayer in the absence or presence of recombinant FL SPARC, or cleaved SPARC at a final concentration of 240 nM. Left panels, representative images of transmigrating cells. Right panel, transmigrating cells were stained with MTT and quantified at 570 nm. CTRL, PBS in cleavage buffer. Data are the mean±SD (n=3); *, p<0.05, **, p<0.01, ***, p<0.001, ANOVA and Bonferroni's post hoc test. Similar results were obtained in two independent experiments. D. Cell invasion. MDA-MB-231 cells were let to invade for 16 h on a Matrigel matrix in the absence or presence of recombinant FL SPARC, or cleaved SPARC at a final concentration of 240 nM. Left panels, representative images of invading cells. Right panel, invading cells were stained with MTT and quantified at 570 nm. CTRL, PBS in cleavage buffer. Data are the mean±SD (n=3); ***, p<0.001, ANOVA and Bonferroni's post hoc test. Similar results were obtained in three independent experiments.

FIG. 6. Effect of FL SPARC and cath-D-induced cleaved SPARC fragments on TNBC cell adhesion. A. Production of Myc/His tagged FL SPARC, and Myc/His tagged 34-, 27-, 16-, 9-, and 6-kDa SPARC fragments. Left panel, equimolar concentrations (240 nM each) of purified Myc/His tagged FL SPARC and SPARC fragments were analysed by SDS-PAGE (17%) and immunoblotting with an anti-Myc antibody (clone 9B11). Right panel, schematic representation of the purified Myc/His tagged SPARC fragments. AC, acidic domain; FL, follistatin-like domain; EC, Ca2+-extracellular binding domain. B. Cell adhesion. MDA-MB-231 cells were let to adhere for 30 min on a fibronectin matrix in the presence of purified Myc/His tagged FL SPARC, or individual Myc/His tagged SPARC fragments (34-, 27-, 16-, 9-, and 6-kDa) at an equimolar final concentration (240 nM each). Upper panels, representative images of adherent cells stained with crystal violet after incubation with the indicated SPARC variants. Lower panel, cell adhesion was quantified as described in FIG. 5A and expressed as percentage relative to the value in control (SPARC-immunodepleted control for each SPARC fragment). Data are the mean±SD of three independent experiments; ***, p<0.001, ANOVA and Bonferroni's post hoc test.

FIG. 7. Effects of the 9-kDa C-terminal SPARC fragment on TNBC cell adhesion, migration, transmigration and invasion. A. Cell adhesion. MDA-MB-231 cells were let to adhere for 30 min on a fibronectin matrix in the presence of recombinant FL SPARC, recombinant cleaved SPARC fragments (cleaved SPARC), or purified 9-kDa C-terminal SPARC fragment at a final concentration of 240 nM. Left panels, representative images of adherent cells stained with crystal violet. Right panel, adhesion was quantified as described in FIG. 5A. Data are the mean±SD (n=3); ***, p<0.001, ANOVA and Bonferroni's post hoc test. CTRL, PBS in cleavage buffer and SPARC-immunodepleted supernatant from the 9-kDa SPARC fragment purification. Similar results were obtained in three independent experiments. B. Cell migration. MDA-MB-231 cells were let to migrate for 16 h on a fibronectin matrix in the absence or presence of FL SPARC, cleaved SPARC fragments, or the 9-kDa C-terminal SPARC fragment at a final concentration of 240 nM. Left panels, representative images of migrating cells stained with crystal violet. Right panel, migration was quantified as described in FIG. 5B. Data are the mean±SD (n=3); ***, p<0.001, ANOVA and Bonferroni's post hoc test. CTRL, PBS in cleavage buffer and SPARC immunodepleted supernatant from the 9-kDa SPARC fragment purification. Similar results were obtained in two independent experiments. C. Endothelial transmigration. MDA-MB-231 cells were let to transmigrate for 16 h through a HUVEC monolayer in the absence or presence of FL SPARC, cleaved SPARC fragments, or the 9-kDa C-terminal SPARC fragment at a final concentration of 240 nM. Left panels, representative images of transmigrating cells. Right panel, transmigrating cells were stained with MTT and quantified by absorbance at 570 nm. Data are the mean±SD (n=3); *, p<0.05, **, p<0.01, ***, p<0.001, ANOVA and Bonferroni's post hoc test. CTRL, PBS in cleavage buffer and SPARC-immunodepleted supernatant from the 9-kDa SPARC fragment purification. Similar results were obtained in two independent experiments. D. Cell invasion. MDA-MB-231 cells were let to invade for 16 h on a Matrigel matrix in the absence or presence of FL SPARC, cleaved SPARC fragments, or the 9-kDa C-terminal SPARC fragment at a final concentration of 240 nM. Left panels, representative images of invading cells stained with crystal violet. Right panel, invading cells were quantified by absorbance at 570 nm. Data are the mean±SD (n=3); *, p<0.05, **, p<0.01, ***, p<0.001, ANOVA and Bonferroni's post hoc test. CTRL, PBS in cleavage buffer and SPARC immunodepleted supernatant from the 9-kDa SPARC fragment purification. Similar results were obtained in two independent experiments.

FIG. 8. Model of the 9-kDa C-terminal SPARC, released by cath-D cleavage, pro-tumour effect on TNBC cells. TNBC-secreted cath-D triggers limited proteolysis of SPARC at the acidic pH of the tumour microenvironment. Among the SPARC fragments cleaved by cath-D, the 9-kDa C-terminal SPARC fragment inhibits TNBC cell adhesion and spreading. This might lead to an intermediate adhesive state, and stimulate TNBC cell migration, endothelial transmigration and invasion.

### EXAMPLE

### Material & Methods

### Antibodies

The rabbit polyclonal anti-SPARC (15274-1-AP) was purchased from Proteintech. The mouse monoclonal anti-human SPARC antibodies (clone AON-5031, sc-73472), the rabbit polyclonal anti-human cath-D antibody (H-75, sc-10725), and the mouse monoclonal anti-human cath-D (clone C-5, sc-377124) were purchased from Santa Cruz Biotechnology. The mouse monoclonal anti-human cath-D antibody (clone 49, #610801) was purchased from BD Transduction Laboratories™, and the goat polyclonal anti-mouse cath-D (AF1029) from R&D systems. The anti-human cath-D antibodies M1G8 and D7E3 were previously described (Beaujouin et al., 2010). The mouse monoclonal anti-tubulin antibody (clone 236-10501, #A11126) was from ThermoFisher Scientific, the mouse monoclonal anti-Myc tag (clone 9B11) from Ozyme, and the rabbit polyclonal anti-β actin antibody (#A2066) from Sigma-Aldrich. The horse anti-mouse immunoglobulin G (IgG)-horseradish peroxidase secondary (#7076), and goat anti-rabbit IgG-HRP secondary antibodies (#7074S) were purchased from Cell Signaling Technology. The donkey anti-goat HRP conjugated (FT-117890) antibody was from Interchim. The Alexa Fluor 488-conjugated anti-rabbit IgG (#Ab150077) was purchased from Abcam, and the Cy3-conjugated anti-mouse IgG (#SA00009.1) from Proteintech. Hoechst 33342 (#FP-BB1340) was from Interchim FluoProbes.

### Cell Lines, Cell Lysis, ELISA, and Western Blotting

Immortalized cath-D-deficient MEFs were provided by C. Peters (University of Freiburg, Freiburg, Germany), and HUVECs by M. Villalba (IRMB, Montpellier). Immortalized cath-D-deficient MEFs stably transfected with empty vector (Ctsd−/−) or the cath-D expression plasmid encoding human pre-pro-cathepsin D (Ctsd−/−cath-D) were previously described¹³. HMFs were provided by J. Loncarek and J. Piette (CRCL Val d'Aurelle-Paul Lamarque, Montpellier, France)¹³. The MDA-MB-231 cell line was previously described (Glondu et al., 2002). The Hs578T, MDA-MB-453 and MDA-MB-468 breast cancer cell lines were obtained from SIRIC Montpellier Cancer. The SUM159 breast cancer cell line was obtained from Asterand (Bioscience, UK). The HEK-293 cell line was kindly provided by A. Maraver (IRCM, Montpellier). Cell lines were cultured in DMEM with 10% foetal calf serum (FCS, GibcoBRL) except the SUM159 cell line that was cultured in RPMI with 10% FCS. Primary murine breast cancer cells were generated from end-stage tumours of CreERT2, Ctsdfl/fl; MMTV-PyMT mice as described previously⁴⁰. All animal procedures were approved by the legal authorities and ethics committee at the regional council Freiburg (registration numbers G14/18 and G18/38) and were performed in accordance with the German law for animal welfare. PyMT cells were cultured in DMEM/F12 medium supplemented with 10% FCS, 2 mM L-glutamine, and 1% Penicillin-Streptomycin at 37° C. with 5% CO2. 3 μM 4-hydroxytamoxifen (OH-Tam, Sigma Aldrich) was added to induce Cre-mediated recombination in the mouse Ctsd gene resulting in a premature stop codon. Cell lysates were harvested in lysis buffer (50 mM HEPES [pH 7.5], 150 mM NaCl, 10% glycerol, 1% Triton X-100, 1.5 mM MgCl2, 1 mM EGTA) supplemented with cOmplete™ protease and phosphatase inhibitor Cocktail (Roche, Switzerland) at 4° C. for 20 min, and centrifuged at 13 000×g at 4° C. for 10 min. Protein concentration was determined using the DC protein assay (Bio-Rad). Cath-D was quantified in TNBC and PDX cytosols by sandwich ELISA, after coating with the D7E3 antibody (200 ng/well in PBS) and with the HRP-conjugated M1G8 antibody (1/80), and using recombinant cath-D (1.25-15 ng/ml), as previously described (Ashraf et al., 2019). TNBC cytosols were previously prepared and frozen (Saadoun et al, 2014). For western blotting, proteins were separated on 13.5% SDS PAGE and analysed by immunoblotting.

### SPARC Cleavage by Cath-D In Vitro and in Cellulo

Recombinant 52-kDa pro-cath-D (4 μM; R&D Systems) was self-activated to 51-kDa pseudo-cath-D in 0.1 M Na-acetate buffer (pH 3.5), 0.2 M NaCl at 37° C. for 15 min, as previously described¹⁷. Recombinant SPARC (1 μM; R&D Systems) was incubated with self-activated pseudo-cath-D (5 nM) at 37° C. at different pH values in cleavage buffer [34 mM Britton-Robinson buffer in the presence of phosphatidylcholine (0.12 mM; Sigma-Aldrich) and cardiolipin (0.05 mM; Sigma-Aldrich) with or without pepstatin A (2 μM; Sigma-Aldrich]. Cleaved SPARC peptides were separated by 13.5% or 17% SDS PAGE and analysed by immunoblotting or silver staining (GE Healthcare Life Sciences), respectively. For in cellulo SPARC cleavage, 200, 000 MDA-MB-231 cells were plated with 100 000 HMF cells in T25 cell culture flasks. After 24 h, culture medium was changed. Conditioned medium from co-cultured MDA-MB-231 cells and HMF was obtained by adding DMEM without sodium bicarbonate buffered with 50 mM HEPES buffer (pH 7.5) and without FCS for 24 h. The 24 h conditioned medium was then incubated, with or without pepstatin A (12.5 μM), at 37° C. in cleavage buffer. Then, proteins in medium (40 μl) were separated by 13.5% SDS-PAGE and analysed by immunoblotting. In other in cellulo SPARC cleavage, 200, 000 Hs578T, SUM159 or PyMT cells were incubated in DMEM without sodium bicarbonate buffered with 50 mM HEPES buffer (pH 7.5) and without FCS for 24 h, and the conditioned medium was analysed as described above.

### Identification of Cath-D-Generated Fragments by ATOMS

Recombinant SPARC (4 μM, 6 μg) was incubated with self-activated 51-kDa or mature 34+14-kDa cath-D (200 nM) in 100 mM Na-acetate buffer (pH 5.9)/0.2 M NaCl with or without pepstatin A (200 μM; Sigma-Aldrich) in the presence of phosphatidylcholine (0.12 mM; Sigma-Aldrich) and cardiolipin (0.05 mM; Sigma-Aldrich) at 37° C. for 15 min. SPARC cleavage was analysed by 13.5% SDS PAGE and silver staining (GE Healthcare Life Sciences). The corresponding samples with/without pepstatin A (5 μg) were then processed for iTRAQ-ATOMS, as previously described (Delolme et al, 2015). Briefly, samples were denatured in 2.5 M guanidine hydrochloride and 0.25 M HEPES pH 8.0 at 65° C. for 15 min, reduced with 1 mM TCEP at 65° C. for 45 min, and alkylated with iodoacetamide at room temperature in the dark for 30 min. After iTRAQ labelling in DMSO, the two samples with/without pepstatin A were mixed and precipitated with eight volumes of freezer-cold acetone and one volume of freezer-cold methanol. The pellet was washed extensively with cold methanol, dried and resuspended in 5 μl of 50 mM NaOH. The pH was adjusted to 8 with 1.8 M HEPES pH 8.0, and the sample was digested at 37° C. with sequencing-grade trypsin (Promega; 1:50 protease:protein w/w ratio) or at 25° C. with Glu-C (Promega; 1:20 protease:protein w/w ratio) overnight. After desalting on a C18 column (Pierce), the sample was analysed by LC-MS/MS on a Q-Exactive HF mass spectrometer operated with the Xcalibur software (version 4.0) and equipped with a RSLC Ultimate 3000 nanoLC system (Thermo Scientific), as previously described⁴¹. Data files were analysed with Proteome Discover 1.4 using the MASCOT (2.2 version) algorithm against the human protein database (SwissProt release 2017-01, 40500 entries including reverse decoy database). Precursor mass tolerance was set at 10 ppm and fragment mass tolerance was set at 0.02 Da, and up to 2 missed cleavages were allowed. Oxidation (M), Deamidation (NQ), acetylation (Protein N-terminus), and iTRAQ 8Plex (N-term, K) were set as variable modifications, and carbamidomethylation (C) as fixed modification. Peptides and proteins were filtered using Percolator and a false discovery rate (FDR) of 1%. Peptides with N-terminal iTRAQ labelling were manually validated. Quantification was performed with the Reporter Ions Quantifier node. The peak integration was set to the Most Confidence Centroid with 20 ppm Integration Mass Tolerance on the reporter ions. The cath-D without pepstatin A/cath-D with pepstatin A ratios were calculated and ratios showing at least a two-fold change are conserved in Table 2 except for peptides corresponding to the mature N-Terminus.

### Secretome Preparation

Secretomes from Ctsd−/− and Ctsd−/−cath-D cells were prepared as previously described (Laurent-Matha et al., 2012). Briefly, cells were washed extensively with phenol red-free, serum-free medium to remove serum proteins and grown overnight in phenol red-free, serum-free medium. Conditioned medium was immediately incubated with protease inhibitors (1 mM EDTA, protease inhibitor cocktail (Complete; Roche Applied Science)), clarified by centrifugation (500 g for 5 min; 8,000 g for 30 min) and filtered (0.45 μM). Proteins present in conditioned medium in 50 mM HEPES (pH 7.5) were then concentrated to 2 mg/ml through Amicon filters (3 kDa cut-off, Millipore). To prepare secretomes from MDA-MB-231/HMF co-cultures, cells (ratio 1:5, respectively) were plated in 150 mm Petri dishes in DMEM with 10% FCS. At a 90% confluence, MDA-MB-231/HMF cells were washed extensively as described above. The 24 h-conditioned medium in 50 mM HEPES (pH 7.5) was then concentrated to 0.2 mg/ml through Amicon filters (3 kDa cut-off, Millipore), and incubated in cleavage buffer with or without pepstatin A (12.5 μM) at pH 5.5 and at 37° for 60 min. Samples were concentrated by TCA/acetone precipitation⁴¹.

### Mass Spectrometry Analysis of Protein N-Termini in Cell Culture Samples (TAILS)

Enrichment of N-terminal peptides by TAILS (Kleifeld et al., 2010) by isotopic labelling of proteins with iTRAQ reagents (Kleifeld et al., 2011) was previously described for Ctsd−/− and Ctsd−/−cath-D¹⁷. Briefly, peptides were pre-fractionated by strong cation exchange chromatography and analysed by LC-MS/MS (QSTAR XL, Applied Biosystems). Peptides were identified at the 95% confidence level from the human UniProtKB/Swissprot protein database using two search engines, MASCOT v2.3 (Matrix Science) and X! TANDEM⁴², in conjunction with PeptideProphet as implemented in the Trans Proteomic Pipeline v4.3. Search parameters were: Semi-ArgC peptides with up to two missed cleavages, 0.4 Da precursor ion mass tolerance, 0.4 Da fragment ion mass tolerance, carboxyamidomethylation of cysteine residues, and iTRAQ labelling of lysine e-amines as fixed modifications, and peptide N-terminal iTRAQ labelling, peptide N-terminal acetylation and Met oxidation as variable modifications. Results from both searches were combined using an in-house software script⁴³. Peptides needed to have an Arg residue at their C-terminus and an iTRAQ reporter ion intensity of >30 in at least one of the compared channels. N-terminal peptides with significant changes between conditions were identified by calculating the log2 of the intensity ratios, correcting the mean of all ratios, and applying a 3-fold change cut-off (mean-corrected log2>1.58 or <−1.58). The abundance of N-terminal peptides was visualized using the raincloud plot R tool (Allen et al, 2019).

For co-cultured MDA-MB-231/HMF cells, TMT labels (126, 127N, 127C, 128N; TMT 10-plex kit 90110 from Thermo Scientific) dissolved in DMSO were added in a 1:5 (total protein/TMT label) mass ratio to one of the four samples (60 μg of total protein per condition) for 60 min. Labelling reactions were stopped with 5% hydroxylamine (Sigma) for 30 min, and the four samples were mixed and precipitated with cold methanol/acetone (8:1) (v/v). After two washes with cold methanol, the pellet was resuspended in 100 mM HEPES at pH 8 at a final protein concentration of 2 mg/ml and digested with trypsin (trypsin/total protein (1:100); Trypsin V511A, Promega) overnight. N-terminal peptide enrichment was performed on the digested sample by removing the internal tryptic peptides with a 1:5 mass excess of dialyzed HPG-ALD polymer, desalted with a C18 spin column (Thermo Fisher Scientific). The eluate fraction was freeze-dried, resuspended in 0.1% FA and analysed by LC-MS/MS on a Q-Exactive HF mass spectrometer, as described above for ATOMS experiments except that the SwissProt 2019-12 Homo sapiens database release was used and that iTRAQ 8-plex was replaced by TMT 10-plex in the list of variable modifications. The ratios without pepstatin A/with pepstatin A were calculated for the two time points (0 min and 60 min) and ratios at 60 min were normalized to the ratios at 0 min. Only peptides with N-terminal TMT labelling and ratios showing at least a two-fold change are indicated in Table 2.

### Study Approval

For TMA, TNBC samples were provided by the biological resource centre (Biobank number BB-0033-00059) after approval by the Montpellier Cancer Institute Institutional Review Board, following the Ethics and Legal national French regulations for patient information and consent. For TNBC cytosols, patient samples were processed according to the French Public Health Code (law n° 2004-800, articles L. 1243-4 and R. 1243-61). The biological resources centre has been authorized (authorization number: AC-2008-700; Val d'Aurelle, ICM, Montpellier) to deliver human samples for scientific research. All patients were informed before surgery that their surgical specimens might be used for research purposes. The study approval for PDXs was previously published⁴³.

### Construction of Tissue Microarrays

Tumour tissue blocks with enough material at gross inspection were selected from the Biological Resource Centre. After haematoxylin-eosin-safranin (HES) staining, the presence of tumour tissue in sections was evaluated by a pathologist. Two representative tumour areas, to be used for the construction of the TMAs, were identified on each slide. A manual arraying instrument (Manual Tissue Arrayer 1, Beecher Instruments, Sun Prairie, WI, USA) was used to extract two malignant cores (1 mm in diameter) from the two selected areas. When possible, normal breast epithelium was also sampled as internal control. After arraying completion, 4 μm sections were cut from the TMA blocks. One section was stained with HES and the others were used for IHC.

### TMA Immunohistochemistry

For SPARC and cath-D immunostaining, serial tumour sections from a TNBC TMA were incubated with 0.2 μg/ml anti-human SPARC mouse monoclonal antibody (clone AON-5031) for 30 min or with 0.4 μg/ml anti-human cath-D mouse monoclonal antibody (clone C-5) for 20 min after heat-induced antigen retrieval with the PTLink pre-treatment (Dako) and the High pH Buffer (Dako) and endogenous peroxidase quenching with Flex Peroxidase Block (Dako). After two rinses in EnVision™ Flex Wash buffer (Dako), sections were incubated with a HRP-labelled polymer coupled to a secondary anti-mouse antibody (Flex® system, Dako) for 20 min, followed by incubation with 3,3′ -diaminobenzidine as chromogen. Sections were counterstained with Flex Hematoxylin (Dako) and mounted after dehydration. Sections were analysed independently by two experienced pathologists, both blinded to the tumour characteristics and patient outcomes at the time of scoring. SPARC signal was scored as low (<50%), or high (>50%), and cath-D signal was scored as absent, low, medium (<50%), high, or very high (>50%) in cancer and stromal cells.

### Fluorescence Microscopy

Paraffin-embedded PDX1995 tissue sections were deparaffined, rehydrated, rinsed, and saturated in PBS with 5% FCS at 4° C. overnight. Sections were co-incubated with 1.2 μg/ml anti-SPARC rabbit polyclonal antibody (Proteintech) and 0.4 μg/ml anti-cath-D mouse monoclonal antibody (clone C-5) followed by co-incubation with AlexaFluor 488-conjugated anti-rabbit IgG (1/400) and a Cy3-conjugated anti-mouse IgG (1/500). Nuclei were stained with 0.5 μg/ml Hoechst 33342. Sections were then imaged with a 63×Plan-Apochromat objective on z stacks with a Zeiss Axio Imager light microscope equipped with Apotome to eliminate out-of-focus fluorescence. For co-staining of SPARC/cath-D, series of three optical sections (0.25 μm thick) were collected and projected onto a single plane.

### siRNA Transfection

The siRNA duplex (21 nucleotides) against human cath-D siRNA (ID 4180) was purchased from Ambion (Austin, TX), and the firefly luciferase (Luc) siRNA was synthesized by MWG Biotech S.A (Bach et al, 2015). MDA-MB-231 cells in 6-well plates were transiently transfected with 4 μg human cath-D or Luc siRNA using Lipofectamine 2000 (Invitrogen). At 48 h post-transfection, 200 000 siRNA-transfected MDA-MB-231 cells were plated with 100 000 HMFs in T25 cell culture flasks for co-culture experiments.

### RT-qPCR

For gene expression analysis, fresh tumour tissues were homogenized in an Ultra-Turrax. RNA was isolated using the RNeasy Mini Kit (Qiagen, Hilden, Germany) and 1 μg of total RNA was reverse transcribed using the iScript™ cDNA Synthesis Kit (Bio-Rad, Feldkirchen, Germany). Real-time PCR was performed using Platinum SYBR Green qPCR Super Mix-UDG (Life Technologies, Darmstadt, Germany) on a CFX96 real-time PCR machine (Bio-Rad).

### Expression and Purification of Recombinant Proteins

The cDNA encoding human SPARC (303 amino acids according to the GenBank reference NP_003109) and its truncated fragments were PCR-amplified using the pcDNA3.1-SPARC plasmid as template (Fenouille et al, 2011), cloned into pGEM®-T Easy Vector (Promega), and then into the pSec-Tag2/hygroA vector (Thermo Fisher Scientific) by Not I digestion. Orientation and sequence were verified. Human embryonic kidney 293 (HEK-293T) cells were stably transfected with the vectors using Lipofectamine 2000 (Invitrogen) according to the manufacturer's instructions, and were selected with 400 μg/ml hygromycin B Gold™ (Invivogen). The recombinant His-tagged proteins were purified from cell lysates on a nickel-chelating column (Ni-nitrilotriacetic acid agarose; His-select high flow nickel affinity gel; Sigma-Aldrich), as described previously (Alcaraz et al., 2014). The isolated recombinant proteins were analysed by western blotting using anti-mouse Myc (clone 9B11) and anti-SPARC (clone AON-5031) antibodies and quantified using the Image J densitometric software (National Institutes of Health). To immunodeplete purified SPARC or its fragments, protein supernatants were incubated with an anti-Myc antibody (clone 9B11) overnight and protein G-Sepharose at 4° C. for 4 h, and supernatants were analysed by immunoblotting to validate SPARC depletion. SPARC-immunodepleted supernatants were used as internal controls in the biological assays.

### Cell Adhesion, Migration, Endothelial Transmigration and Invasion Assays

Adhesion of MDA-MB-231 cells was assessed as described (Alcaraz et al., 2014). Briefly, 96-well plates were coated with fibronectin (10 μg/ml; sc-29011; Santa Cruz Biotechnology) at 4° C. overnight, and saturated with 1% BSA in PBS. MDA-MB-231 cells were detached with HyQTase (HyClone), washed in DMEM without FCS, and 1.5 105 cells were pre-incubated or not with SPARC or its cleaved fragments at room temperature for 10 min. Cells (5 104 cells) were plated and left in serum-free medium at 37° C. for 30 min. Non-adherent cells were removed by floatation on a dense Percoll solution containing 3.33% NaCl (1.10 g/l), and adherent cells were fixed (10% [vol/vol] glutaraldehyde) using the buoyancy method⁴⁵. Cells were stained with 0.1% crystal violet, and absorbance was measured at 570 nm. For migration assays, 8-μm pore Transwell inserts in 24-well plates (polyvinyl pyrrolidone-free polycarbonate filter) (Corning Inc., Corning, NY, USA) were coated with 10 μg/ml fibronectin (500 ng) at 4° C. for 24 h. For invasion assays, 8-μm pore Transwell inserts were coated with Matrigel (100 μg, Corning). MDA-MB-231 cells (2 105 cells) were pre-incubated or not with SPARC or its cleaved fragments at room temperature for 10 min, and then plated (5 104 cells/well) in FCS-free DMEM on the coated insert in the upper chamber. For transmigration assay, 105 HUVECs were plated in the upper chamber of a gelatine-coated Transwell insert and grown in complete endothelial medium to confluence, as previously described²⁴. The endothelial monolayer was then incubated with human TNFα (10 ng/ml; PeproTech) for 16 h. MDA-MB-231 cells (3 105 cells), pre-incubated or not with SPARC or its cleaved fragments at room temperature for 10 min, were then plated (105 cells/well) in FCS-free DMEM on top of the endothelial monolayer. In these different assays, DMEM supplemented with 10% FCS was used as chemoattractant in the bottom chamber. After 16 h, non-migrating/non-invading/non-transmigrating cells on the apical side of each insert were scraped off with a cotton swab, and migration, invasion and transmigration were analysed with two methods: (1) migrating/invading/transmigrating cells were fixed in methanol, stained with 0.1% crystal violet for 30 min, rinsed in water, and imaged with an optical microscopy. Two images of the pre-set field per insert were captured (×100); (2) migrating/invading/transmigrating cells were incubated with 3-(4,5-dimethylthiazol-2-yl)-2,5-diphenyltetrazolium bromide (MTT; 5 mg/ml, 1/10 volume; Sigma-Aldrich) added to the culture medium at 37° C. for 4 h. Then, the culture medium/MTT solution was removed and centrifuged at 10 000 rpm for 5 min. After centrifugation, cell pellets were suspended in DMSO. Concomitantly, 300 μl of DMSO was added to each well and thoroughly mixed for 5 min. The optical density values of stained cells (cell pellet and corresponding well) were measured using a microplate reader at 570 nm.

### Results

### Identification of SPARC as an Extracellular Protein Affected by Cath ID Deficiency

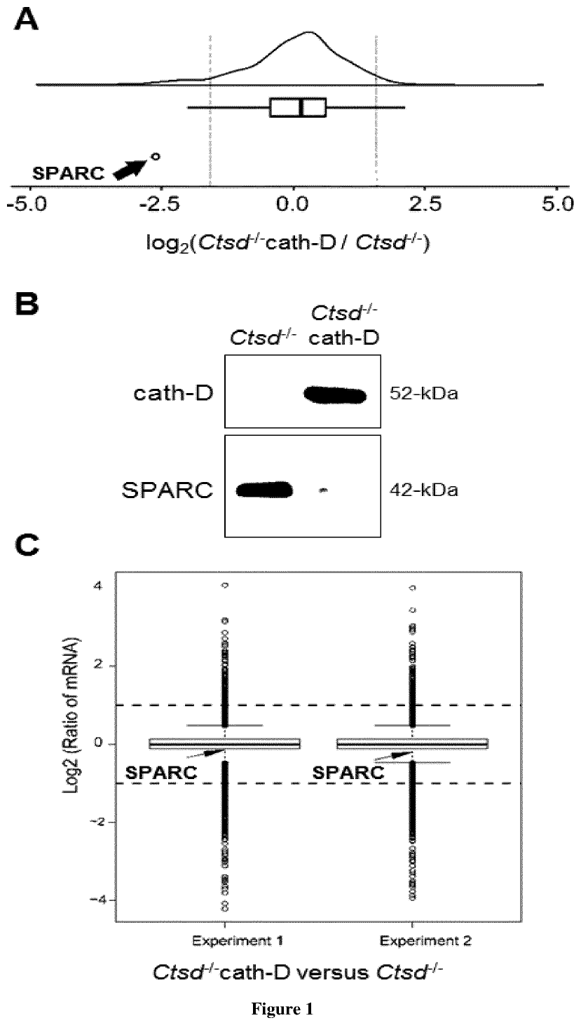

Using the TAILS approach, we previously analysed the secretome of immortalized Ctsd−/− MEFs stably transfected with empty vector (Ctsd−/−) or a human cath-D (Ctsd−/−cath-D) plasmid to determine cath-D effect on extracellular protein processing¹⁷. We noticed that the SPARC peptide LDSELTEFPLR (SEQ ID NO:1) [156-166] was 5.2-fold less abundant in the secretome of Ctsd−/−cath-D MEFs than of Ctsd−/− MEFs (FIG. 1A). To determine whether SPARC is a putative cath-D substrate, we first confirmed that SPARC protein level was reduced in the Ctsd−/−cath-D secretome compared with the Ctsd−/− secretome (FIG. 1B). Transcriptome analysis of Ctsd−/− and Ctsd−/−cath-D MEFs, as previously published¹⁷ showed that SPARC reduction in the Ctsd−/−cath-D secretome was not due to Sparc gene downregulation in the presence of cath-D (FIG. 1C). These data showed that SPARC protein level in the extracellular environment is reduced in the presence of cath-D.

### In Vitro, Cath-D Cleaves SPARC Extracellular Ca2+ Binding Domain at Acidic pH

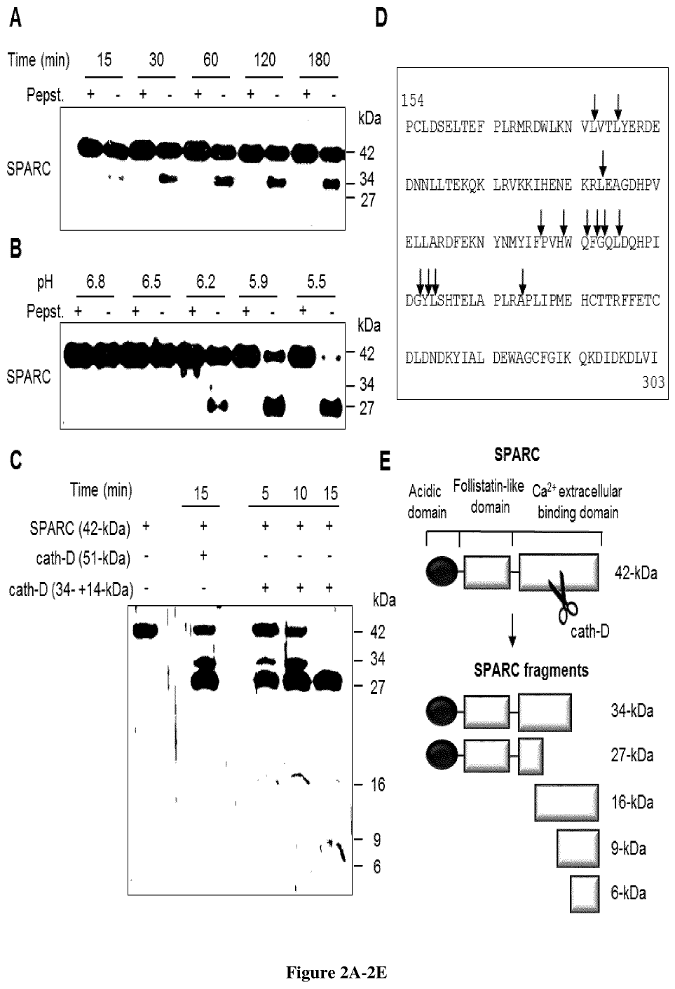

We investigated whether recombinant cath-D can cleave recombinant SPARC in vitro at acidic pH. At pH 5.9, SPARC was hydrolysed by cath-D in a time-dependent manner (FIG. 2A). Moreover, experiments in which pH was gradually reduced from 6.8 to 5.5 showed progressive limited proteolysis of SPARC at lower pH (FIG. 2B). In these two experiments, pepstatin, an aspartic protease inhibitor, inhibited SPARC cleavage by cath-D (FIG. 2A-B). By amino-terminal oriented mass spectrometry of substrates (ATOMS) analysis, we found that SPARC was hydrolysed by the 51-kDa cath-D form exclusively in its extracellular Ca2+ binding domain, releasing five main SPARC fragments (34-, 27, 16, 9-, and 6-kDa) at pH 5.9, detected by silver staining (FIG. 2C-E, Table 2). We detected SPARC cleavage fragments of similar size also after incubation with the fully mature 34+14-kDa cath-D form at pH 5.9 (FIG. 2C-E, Table 2). Thus, in vitro, cath-D triggers the limited proteolysis of SPARC exclusively in its extracellular Ca2+ binding domain in an acidic environment.

### SPARC and Cath-D Expression in TNBC

To study the pathophysiological relevance of the SPARC/cath-D interplay in TNBC, we first assessed the clinical significance of SPARC and CTSD (the gene encoding cath-D) expression in a cohort of 255 patients with TNBC using an online survival analysis⁴⁶. High CTSD mRNA level was significantly associated with shorter recurrence-free survival (HR=1.65 for [1.08-2.53]; p=0.019) (data not shown), as previously observed¹⁵. Similarly, high SPARC mRNA level tended to be associated with shorter recurrence-free survival (HR=1.6 [0.91-2.79]; p=0.097) (data not shown). We then examined SPARC and cath-D expression by immunohistochemistry (IHC) analysis in serial sections of a TNBC Tissue Micro-Array (TMA) (data not shown). Cath-D was expressed mainly in cancer cells, and to a lesser extent in macrophages, fibroblasts and adipocytes in the stroma (data not shown). Conversely, SPARC was expressed mainly in fibroblasts, macrophages and endothelial cells in the tumour stroma, whereas its expression level in cancer cells was variable (data not shown). Next, we analysed SPARC and cath-D expression and secretion in different TNBC cell lines and in human mammary fibroblasts (HMF) (data not shown). Cath-D was expressed by TNBC and HMF cells (data not shown), but was secreted only by TNBC cells (data not shown). Conversely, SPARC was expressed and secreted by HMF, but only by two out of five TNBC cell lines, namely SUM159 and HS578T (data not shown). Finally, we investigated SPARC and cath-D co-localization in a TNBC patient-derived xenograft (PDX B1995) (du Manoir et al, 2014) in which cath-D expression was previously demonstrated¹⁵. Co-labelling with polyclonal anti-SPARC and monoclonal anti-cath-D antibodies showed that SPARC partially co-localized with cath-D in the PDX B1995 microenvironment (data not shown).

Together with previously published data on SPARC^{27-29, 31-33} and cath-D^{2-4, 6,7, 9-14, 16} in BC, our results strongly suggest that it is important to investigate the relationship between SPARC and cath-D that are both co-secreted in the TNBC microenvironment.

### At Acidic pH, Cath ID Secreted by TNBC and Mouse Mammary Cancer Cells Cleaves Fibroblast- and Cancer-Derived SPARC in its Extracellular Ca2+ Binding Domain

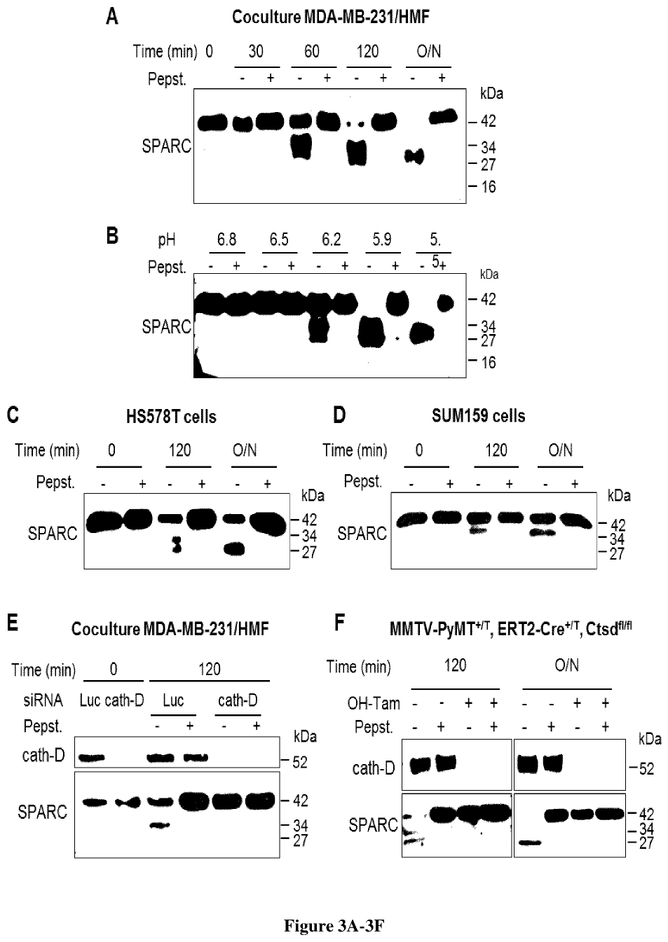

As the tumour extracellular environment is acidic, we then asked whether cath-D can degrade SPARC in the extracellular medium of TNBC cells at low pH. First, we used conditioned medium from cath-D-secreting TNBC MDA-MB-231 cells co-cultured with SPARC-secreting HMFs for 24 h (data not shown). SPARC was hydrolysed in a time-dependent manner in the conditioned medium at pH 5.5 (FIG. 3A). By western blot analysis, we detected mainly the 34-kDa and 27-kDa SPARC fragments, and to a lesser extent, the 16-kDa fragment (FIG. 3A). Pepstatin A inhibited SPARC cleavage, confirming the involvement of secreted aspartic protease proteolytic activity (FIG. 3A). Moreover, TAILS analysis of the secretome in conditioned medium of co-cultured MDA-MB-231/HMF cells at pH 5.5 showed the presence of the five main SPARC fragments (34, 27-, 16-, 9-, and 6-kDa) only in the absence of pepstatin A (Table 2). We then assessed SPARC hydrolysis at different pH (6.8 to 5.5), and found that in the MDA-MB-231/HMF conditioned medium, SPARC was significantly degraded up to pH 6.2 (FIG. 3B), similarly to the results obtained with recombinant proteins (FIG. 2B). In addition, we observed SPARC limited proteolysis also in conditioned medium of TNBC HS578T (FIG. 3C) and TNBC SUM159 cells (FIG. 3D), which secrete both proteins, at pH 5.5. Finally, we did not observe SPARC cleavage at pH 5.5 in conditioned medium from HMFs co-cultured with MDA-MB-231 cells in which CTSD was silenced by RNA interference, indicating that cath-D was responsible for SPARC proteolysis in acidic conditions (FIG. 3E). We confirmed cath-D direct involvement in SPARC processing also by using a mammary cancer cell line derived from tamoxifen-inducible CreERT2, Ctsdfl/fl mice⁴⁷ crossed with the transgenic MMTV-PyMT mouse model of metastatic BC⁴⁸ (FIG. 3F). In the absence of hydroxytamoxifen (OH-Tam), both cath-D and SPARC were secreted by these cells, whereas cath-D expression and secretion were abrogated by incubation with OH-Tam (data not shown). SPARC was hydrolysed in the conditioned medium from this mouse mammary cancer cell line at pH 5.5 only in the absence of OH-Tam when cath-D was secreted (FIG. 3F). These findings demonstrate that cath-D secreted by TNBC and mouse mammary tumour cells cleaves SPARC in its extracellular Ca2+ binding domain at the acidic pH found in the tumour microenvironment.

### SPARC is Cleaved In Vivo in TNBC and Mouse Mammary Tumours

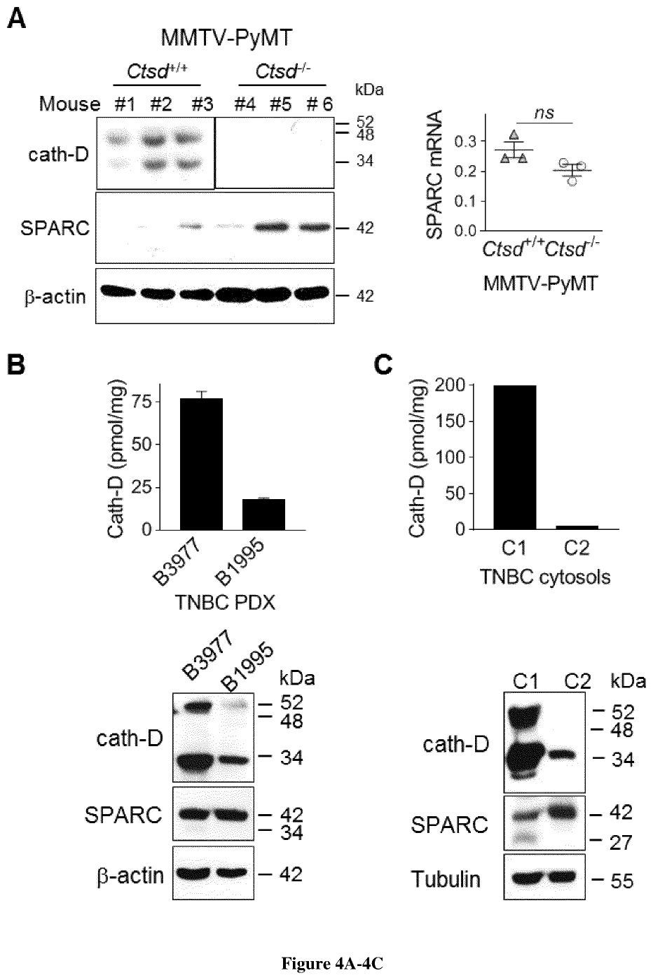

To validate cath-D-dependent SPARC cleavage in vivo, we first analysed FL SPARC protein level and its cleaved fragments in whole cytosols of mammary tumours from MMTV-PyMT cath-D knock-out mice (FIG. 4A). As expected, cath-D was expressed in the cytosol of mammary tumours from MMTV-PyMT, Ctsd+/+ mice, but not from MMTV-PyMT, Ctsd−/− mice (FIG. 4A, left panel). In two of the three tumours from MMTV-PyMT, Ctsd−/− mice, SPARC expression level was much higher than in the three tumours from MMTV-PyMT, Ctsd+/+ mice (FIG. 4A, left panel). Unexpectedly, we could not detect any SPARC cleavage fragment in this transgenic mouse model, certainly due to further SPARC proteolysis in vivo by other proteinases. Nevertheless, SPARC reduction occurred through post-translational mechanisms because SPARC mRNA level was not significantly different in the corresponding MMTV-PyMT, Ctsd+/+ and MMTV-PyMT, Ctsd−/− tumours (FIG. 4A, right panel). We then evaluated the presence of FL SPARC and its cleaved fragments in the whole cytosols from two TNBC PDXs that express cath-D at high and low level, respectively (FIG. 4B, top panel). We detected FL SPARC and its 34-kDa cleaved fragment in PDX B3977 (high cath-D expression), but only FL SPARC in PDX B1995 (low cath-D expression) (FIG. 4B, bottom panel). Finally, we analysed the level of FL SPARC and its cleaved fragments in whole cytosols from two TNBC clinical samples with different cath-D expression levels (FIG. 4C, top panel). The level of FL SPARC was lower in cytosol C1 (TNBC with high cath-D expression) than in cytosol C2 (TNBC with low cath-D expression) (FIG. 4C, bottom panel). Moreover, we detected the 27-kDa cleaved SPARC fragment only in cytosol C1 (high cath-D expression) (FIG. 4C, bottom panel). Overall, these results strongly suggest that SPARC cleavage in its extracellular Ca2+ binding domain may occur in vivo in mammary cancers in the presence of cath-D, although other proteinases may also be involved.

### Cath-D-Induced SPARC Fragments Inhibit TNBC Cell Adhesion and Spreading, and Promote their Motility, Endothelial Transmigration and Invasion

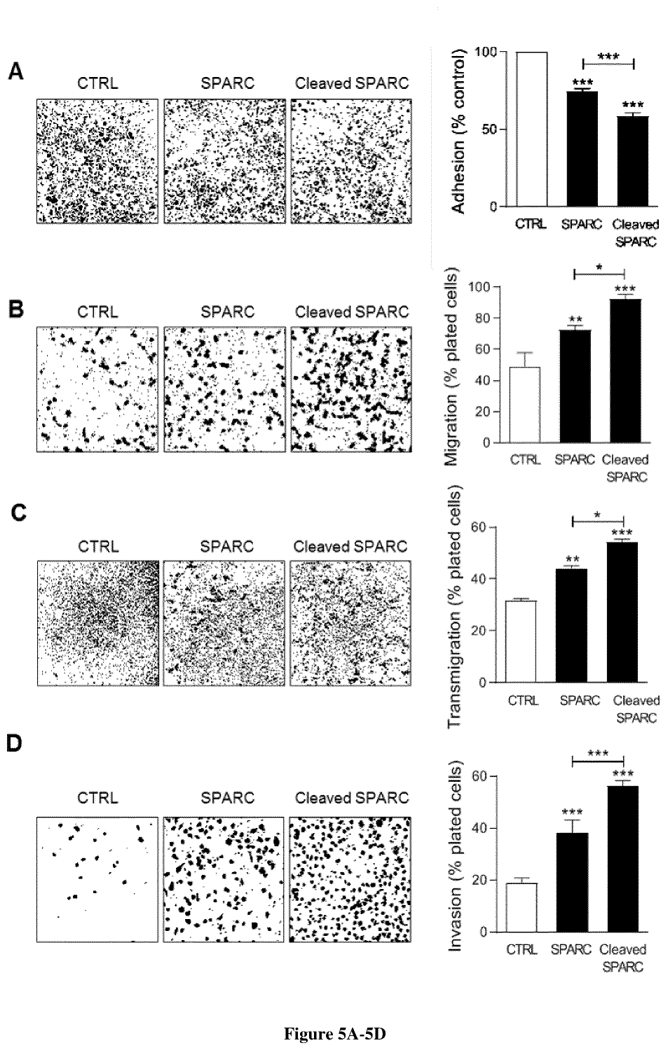

Previous studies reported that FL SPARC and particularly its C-terminal extracellular Ca2+ binding domain can modulate adhesion, spreading, motility, endothelial transmigration, and invasion of cancer and stromal cells^{20, 24, 31, 49}. Therefore, we compared the effect of the cath-D-induced SPARC fragments (mixture of 34+27+16+9+6-kDa fragments) (data not shown) and of FL recombinant SPARC (42-kDa) in MDA-MB-231 cells. Soluble FL SPARC significantly inhibited MDA-MB-231 cell adhesion on fibronectin in a dose-dependent manner (data not shown). After incubation with FL SPARC (final concentration of 10 μg/ml, 240 nM), as previously described²⁴, MDA-MB-231 cell adhesion on fibronectin was reduced by 1.3-fold compared with control (CTRL; untreated) (FIG. 5A; p<0.001). Moreover, FL SPARC inhibition of cell adhesion was similar in Luc- and cath-D-silenced MDA-MB-231 cells, indicating an autonomous effect of SPARC on cell adhesion (data not shown). Incubation of MDA-MB-231 cells with cath-D-induced SPARC fragments (cleaved SPARC) also significantly decreased cell adhesion by 1.7-fold compared with control (CTRL) (FIG. 5A; p<0.001) and by 1.3-fold compared with FL SPARC (FIG. 5A; p<0.001). We also monitored the effects of FL and cleaved SPARC on MDA-MB-231 cell spreading on fibronectin by staining F-actin filaments with phalloidin (data not shown). Both FL SPARC and SPARC cleaved fragments led to a decrease of the cell surface contact area on fibronectin through a peripheral rearrangement of F-actin. Specifically, bundling of actin stress fibres was disrupted and actin microfilaments were redistributed in a peripheral web (data not shown). This suggests a transition to an intermediate state of adhesiveness, previously described for FL SPARC that may favour cell migration and invasion. Incubation with FL and cleaved SPARC fragments decreased the percentage of spread cells by 2.1-fold and 3.8-fold, respectively, compared with control (data not shown). This inhibition was significantly higher (1.8-fold) with cleaved SPARC than FL SPARC (data not shown). Then, cell motility analysis in Boyden chambers showed quite high basal motility of MDA-MB-231 cells, as expected for mesenchymal cells (e.g. 49% of cells passed through the fibronectin-coated filters) (FIG. 5B). Incubation with FL and cleaved SPARC increased MDA-MB-231 cell motility by 1.5-fold and 1.9-fold, respectively, compared with control (FIG. 5B; p<0.01 and p<0.001). Moreover, the effect of cleaved SPARC on cell motility was 1.3-fold higher than that of FL SPARC (FIG. 5B; p<0.05). In the endothelial transmigration assay, FL and cleaved SPARC fragments stimulated MDA-MB-231 migration through primary human umbilical vein endothelial cells (HUVECs) by 1.4-fold and 1.7-fold, respectively, compared with control (FIG. 5C; p<0.01 and p<0.001). The effect of cleaved SPARC was 1.2-fold higher than that of FL SPARC (FIG. 5C; p<0.05). Finally, both FL and cleaved SPARC fragments increased MDA-MB-231 cell invasion through Matrigel-coated filters in Boyden chambers by 2-fold and 3-fold, respectively, compared with control (FIG. 5D; p<0.001). The effect of cleaved SPARC was 1.5-fold higher than that of FL SPARC (FIG. 5D; p<0.001). Altogether, these results indicate that FL SPARC inhibits MDA-MB-231 cell adhesion and spreading, and promotes MDA-MB-231 cell motility, endothelial transmigration, and invasion. These effects were increased by incubation with cath-D-induced SPARC fragments, suggesting that in the TNBC microenvironment, cath-D amplifies SPARC pro-tumour activity through proteolysis of its extracellular Ca2+ binding domain.

### The 9-kDa C-Terminal SPARC Fragment Inhibits TNBC Cell Adhesion and Spreading, and Promotes their Motility, Endothelial Transmigration, and Invasion

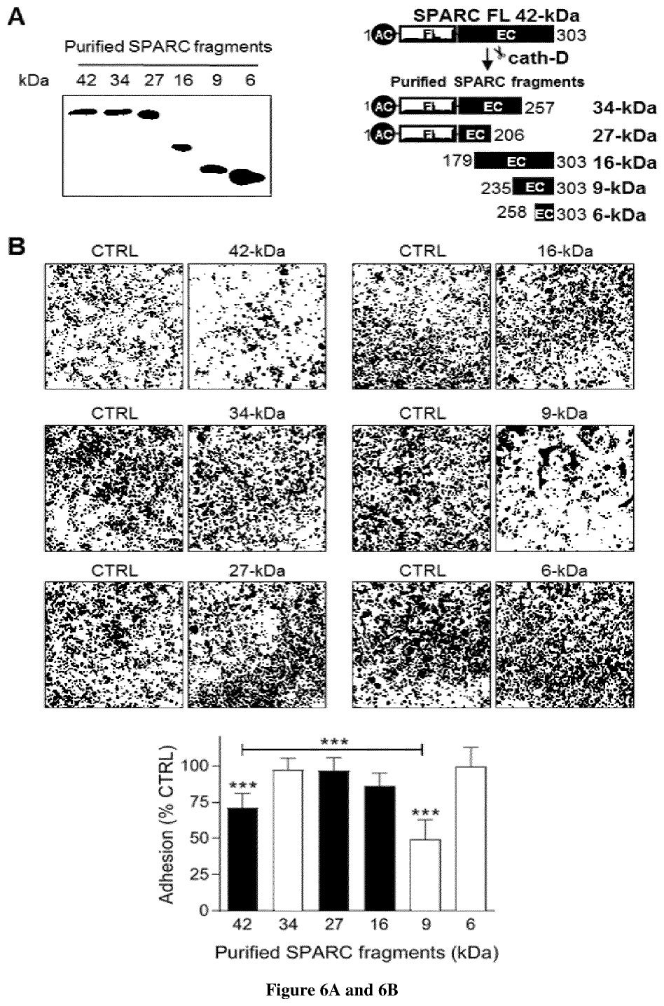

To identify the SPARC domain(s) involved in these functions, we produced FL SPARC and its various cleaved fragments in mammalian cells and purified them, as previously described^{50,51} (FIG. 6A). We first determined which SPARC fragment(s) were involved in the reduction of cell adhesion by incubating MDA-MB-231 cells with equimolar amounts of FL protein and each fragment (FIG. 6B). As before (FIG. 5A), purified FL SPARC (42-kDa) reduced MDA-MB-231 cell adhesion by 1.4-fold compared with control (FIG. 6B; p<0.001). However, among the C-terminal SPARC fragments, only the 9-kDa fragment (amino acids 235-303) significantly decreased MDA-MB-231 cell adhesion by 2-fold compared with control (FIG. 6B; p<0.001), and by 1.4-fold compared with FL SPARC (FIG. 6B; p<0.001). The 9-kDa C-terminal SPARC fragment (amino acids 235-303) contains the two Ca2+ binding sequences of the two EF-hand domains (data not shown), that are involved in focal adhesion disassembly, and are crucial for SPARC-mediated inhibition of adhesion. The 16-kDa C-terminal SPARC fragment (amino acids 179-303) reduced cell adhesion by 1.2-fold (not significant) (FIG. 6B), and the 6-kDa SPARC fragment (amino acids 258-303) had no effect (FIG. 6B). Therefore, among the five cath-D-induced SPARC fragments (FIG. 2E), only the C-terminal 9-kDa fragment could inhibit cell adhesion and more potently than FL SPARC.

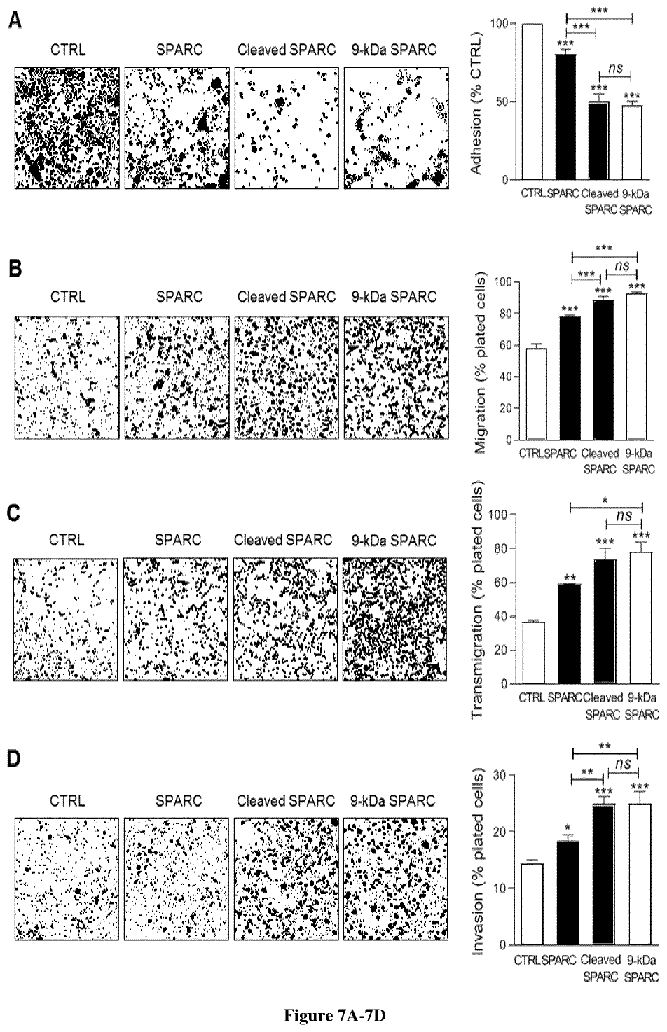

Based on these results, we compared the effects on MDA-MB-231 cell adhesion, spreading, motility, endothelial transmigration and invasion of the 9-kDa C-terminal SPARC fragment, FL SPARC, and the mixture of cath-D cleaved SPARC fragments (34-, 27-, 16-, 9-, and 6-kDa) (FIG. 7). Incubation with the 9-kDa C-terminal SPARC fragment significantly decreased cell adhesion (FIG. 7A, p<0.001) and spreading (data not shown) by 2.1-fold, and 8.4-fold, respectively, compared with control, and significantly increased cell motility by 1.6-fold (FIG. 7B; p<0.001), endothelial transmigration by 2.1-fold (FIG. 7C; p<0.001), and cell invasion by 1.7-fold (FIG. 7D; p<0.001) compared with control. Moreover, the 9-kDa SPARC fragment seemed to induce a transition to an intermediate adhesive state highlighted by the loss of actin-containing stress fibres (data not shown). Conversely, we did not observe any significant difference between the 9-kDa C-terminal SPARC and the cath-D-induced SPARC fragments (FIG. 7).

### Discussion

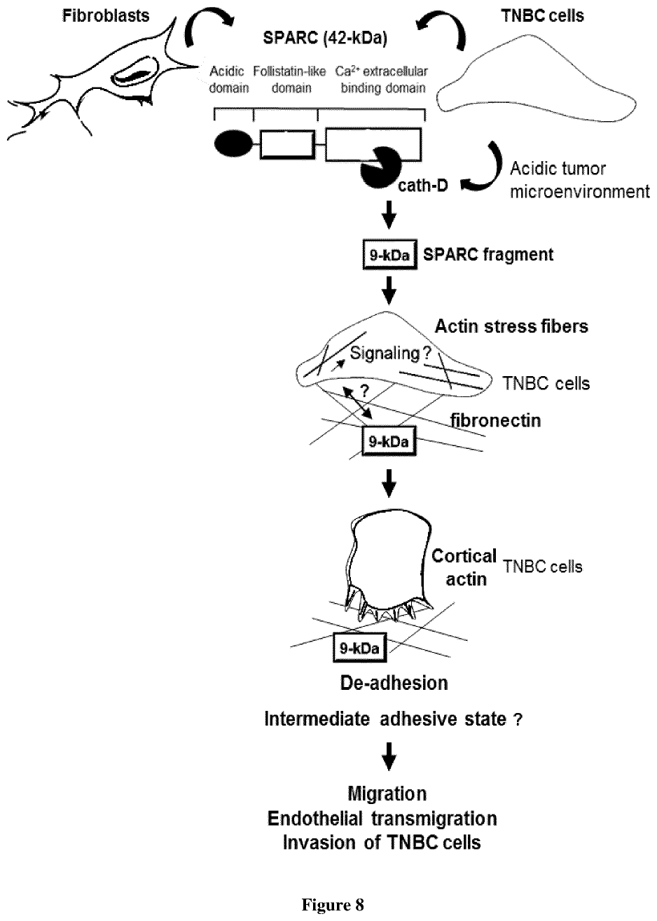

This study shows that cath-D secreted by TNBC cells triggers fibroblast- and cancer-derived SPARC cleavage at the acidic pH of the tumour microenvironment, leading to the production of the bioactive 9-kDa C-terminal SPARC fragment that inhibits cancer cell adhesion and spreading, and stimulates their migration, endothelial transmigration and invasion (FIG. 8). The TAILS analysis of the secretomes of conditioned medium from co-cultured TNBC cells and HMFs revealed that five main SPARC fragments (34-, 27-, 16-, 9-, and 6-kDa) are released in the extracellular environment in a cath-D-dependent manner. Our previous TAILS study showed that cystatin C is a substrate of extracellular cath-D and it is completely degraded by multiple cleavage, highlighting the complexity of the proteolytic cascades that operate in the tumour microenvironment¹⁷. Here, we demonstrate that cath-D triggers also the limited proteolysis of the matricellular protein SPARC in an acidic environment to favour TNBC invasion.

Our current results indicate that cath-D secreted by TNBC cells is part of the proteolytic network in the TNBC acidic microenvironment that generates a bioactive 9-kDa C-terminal fragment of the matricellular protein SPARC with enhanced oncogenic activity. We dissected the molecular mechanisms that link SPARC limited cleavage by cath-D in TNBC microenvironment to the amplified oncogenic activity of a 9-kDa C-terminal fragment of SPARC, highlighting a novel paradigm of alteration of the extracellular milieu of TNBC by proteolysis. Overall, these results indicate that the 9-kDa C-terminal SPARC fragment is an attractive target for cancer therapies in TNBC, and open the way for developing novel targeted therapies against bioactive fragments from matricellular proteins, for both restructuring the surrounding microenvironment and reducing tumorigenesis

### REFERENCES

Throughout this application, various references describe the state of the art to which this invention pertains. The disclosures of these references are hereby incorporated by reference into the present disclosure.
1. Bianchini G, Balko J M, Mayer I A, Sanders M E, Gianni L. Triple-negative breast cancer: challenges and opportunities of a heterogeneous disease. Nature reviews Clinical oncology 13, 674-690 (2016).
2. Ferrandina G, Scambia G, Bardelli F, Benedetti Panici P, Mancuso S, Messori A. Relationship between cathepsin-D content and disease-free survival in node-negative breast cancer patients: a meta-analysis. Br J Cancer 76, 661-666 (1997).
3. Foekens J A, Look M P, Bolt-de Vries J, Meijer-van Gelder M E, van Putten W L, Klijn J G. Cathepsin-D in primary breast cancer: prognostic evaluation involving 2810 patients. Br J Cancer 79, 300-307 (1999).
4. Kang J, et al. Prognostic role of high cathepsin D expression in breast cancer: a systematic review and meta-analysis. Ther Adv Med Oncol 12, 1758835920927838 (2020).
5. Huang L, Liu Z, Chen S, Liu Y, Shao Z. A prognostic model for triple-negative breast cancer patients based on node status, cathepsin-D and Ki-67 index. PloS one 8, e83081 (2013).
6. Mansouri H, et al. Co-Expression of Androgen Receptor and Cathepsin D Defines a Triple-Negative Breast Cancer Subgroup with Poorer Overall Survival. Cancers (Basel) 12, (2020).
7. Vignon F, Capony F, Chambon M, Freiss G, Garcia M, Rochefort H. Autocrine growth stimulation of the MCF 7 breast cancer cells by the estrogen-regulated 52 K protein. Endocrinology 118, 1537-1545 (1986).
8. Hasilik A, von Figura K, Conzelmann E, Nehrkorn H, Sandhoff K. Lysosomal enzyme precursors in human fibroblasts. Activation of cathepsin D precursor in vitro and activity of beta-hexosaminidase A precursor towards ganglioside GM2. Eur J Biochem 125, 317-321 (1982).
9. Berchem G, et al. Cathepsin-D affects multiple tumor progression steps in vivo: proliferation, angiogenesis and apoptosis. Oncogene 21, 5951-5955 (2002).
10. Glondu M, Coopman P, Laurent-Matha V, Garcia M, Rochefort H, Liaudet-Coopman E. A mutated cathepsin-D devoid of its catalytic activity stimulates the growth of cancer cells. Oncogene 20, 6920-6929 (2001).
11. Glondu M, Liaudet-Coopman E, Derocq D, Platet N, Rochefort H, Garcia M. Down-regulation of cathepsin-D expression by antisense gene transfer inhibits tumor growth and experimental lung metastasis of human breast cancer cells. Oncogene 21, 5127-5134 (2002).
12. Beaujouin M, et al. Pro-cathepsin D interacts with the extracellular domain of the beta chain of LRP1 and promotes LRP1-dependent fibroblast outgrowth. Journal of cell science 123, 3336-3346 (2010).
13. Laurent-Matha V, et al. Catalytically inactive human cathepsin D triggers fibroblast invasive growth. J Cell Blot 168, 489-499 (2005).
14. Hu L, Roth J M, Brooks P, Luty J, Karpatkin S. Thrombin up-regulates cathepsin D which enhances angiogenesis, growth, and metastasis. Cancer Res 68, 4666-4673 (2008).
15. Ashraf Y, et al. Immunotherapy of triple-negative breast cancer with cathepsin D-targeting antibodies. J Immunother Cancer 7, 29 (2019).
16. Kleifeld O, et al. Identifying and quantifying proteolytic events and the natural N terminome by terminal amine isotopic labeling of substrates. Nat Protoc 6, 1578-1611 (2011).
17. Laurent-Matha V, et al. Proteolysis of cystatin C by cathepsin D in the breast cancer microenvironment. FASEB journal: official publication of the Federation of American Societies for Experimental Biology, (2012).
18. Brekken R A, Sage E H. SPARC, a matricellular protein: at the crossroads of cell-matrix. Matrix Biol 19, 569-580 (2000).
19. Lane T F, Sage E H. The biology of SPARC, a protein that modulates cell-matrix interactions. FASEB journal: official publication of the Federation of American Societies for Experimental Biology 8, 163-173 (1994). Murphy-Ullrich & Sage, 2014;
20. Murphy-Ulrich J E, Sage E H. Revisiting the matricellular concept. Matrix Biol. 2014 July; 37:1-14.
21. Sage E H, Bornstein P. Extracellular proteins that modulate cell-matrix interactions. SPARC, tenascin, and thrombospondin. The Journal of biological chemistry 266, 14831-14834 (1991).
22. Barth P J, Moll R, Ramaswamy A. Stromal remodeling and SPARC (secreted protein acid rich in cysteine) expression in invasive ductal carcinomas of the breast. Virchows Archiv: an international journal of pathology 446, 532-536 (2005).
23. Hsiao Y H, Lien H C, Hwa H L, Kuo W H, Chang K J, Hsieh F J. SPARC (osteonectin) in breast tumors of different histologic types and its role in the outcome of invasive ductal carcinoma. Breast J 16, 305-308 (2010).
24. Tichet M, et al. Tumour-derived SPARC drives vascular permeability and extravasation through endothelial VCAM1 signalling to promote metastasis. Nature communications 6, 6993 (2015).
25. Nagaraju G P, Dontula R, El-Rayes BF, Lakka S S. Molecular mechanisms underlying the divergent roles of SPARC in human carcinogenesis. Carcinogenesis 35, 967-973 (2014).
26. Podhajcer O L, Benedetti L G, Girotti M R, Prada F, Salvatierra E, Llera A S. The role of the matricellular protein SPARC in the dynamic interaction between the tumor and the host. Cancer metastasis reviews 27, 691-705 (2008).
27. Briggs J, Chamboredon S, Castellazzi M, Kerry J A, Bos T J. Transcriptional upregulation of SPARC, in response to c-Jun overexpression, contributes to increased motility and invasion of MCF7 breast cancer cells. Oncogene 21, 7077-7091 (2002).
28. Guttlein L N, et al. Predictive Outcomes for HER2-enriched Cancer Using Growth and Metastasis Signatures Driven By SPARC. Mol Cancer Res 15, 304-316 (2017).
29. Hsiao Y H, Lien H C, Hwa H L, Kuo W H, Chang K J, Hsieh F J. SPARC (osteonectin) in breast tumors of different histologic types and its role in the outcome of invasive ductal carcinoma. Breast J 16, 305-308 (2010).
30. McQuerry J A, et al. Pathway activity profiling of growth factor receptor network and stemness pathways differentiates metaplastic breast cancer histological subtypes. BMC cancer 19, 881 (2019).
31. Sangaletti S, et al. Mesenchymal Transition of High-Grade Breast Carcinomas Depends on Extracellular Matrix Control of Myeloid Suppressor Cell Activity. Cell Rep 17, 233-248 (2016).
32. Watkins G, Douglas-Jones A, Bryce R, Mansel R E, Jiang W G. Increased levels of SPARC (osteonectin) in human breast cancer tissues and its association with clinical outcomes. Prostaglandins Leukot Essent Fatty Acids 72, 267-272 (2005).
33. Zhu A, et al. SPARC overexpression in primary tumors correlates with disease recurrence and overall survival in patients with triple negative breast cancer. Oncotarget 7, 76628-76634 (2016).
34. Dhanesuan N, Sharp J A, Blick T, Price J T, Thompson E W. Doxycycline-inducible expression of SPARC/Osteonectin/BM40 in MDA-MB-231 human breast cancer cells results in growth inhibition. Breast cancer research and treatment 75, 73-85 (2002).
35. Koblinski J E, et al. Endogenous osteonectin/SPARC/BM-40 expression inhibits MDA-MB-231 breast cancer cell metastasis. Cancer Res 65, 7370-7377 (2005).
36. Ma J, et al. SPARC inhibits breast cancer bone metastasis and may be a clinical therapeutic target. Oncol Lett 14, 5876-5882 (2017).
37. Bradshaw A D, Sage E H. SPARC, a matricellular protein that functions in cellular differentiation and tissue response to injury. J Clin Invest 107, 1049-1054 (2001).
38. Clark C J, Sage E H. A prototypic matricellular protein in the tumor microenvironment—where there's SPARC, there's fire. J Cell Biochem 104, 721-732 (2008).
39. Murphy-Ullrich J E, Sage E H. Revisiting the matricellular concept. Matrix Biol 37, 1-14 (2014).
40. Sevenich L, et al. Transgenic expression of human cathepsin B promotes progression and metastasis of polyoma-middle-T-induced breast cancer in mice. Oncogene 30, 54-64 (2011).
41. Heumuller S E, et al. C-terminal proteolysis of the collagen VI alpha3 chain by BMP-1 and proprotein convertase(s) releases endotrophin in fragments of different sizes. The Journal of biological chemistry 294, 13769-13780 (2019).
42. Craig R, Beavis R C. TANDEM: matching proteins with tandem mass spectra. Bioinformatics 20, 1466-1467 (2004).
43. Keller U, Prudova A, Gioia M, Butler G S, Overall C M. A statistics-based platform for quantitative N-terminome analysis and identification of protease cleavage products. Mol Cell Proteomics 9, 912-927 (2010).
44. du Manoir S, et al. Breast tumor PDXs are genetically plastic and correspond to a subset of aggressive cancers prone to relapse. Molecular oncology 8, 431-443 (2014).
45. Goodwin A E, Pauli B U. A new adhesion assay using buoyancy to remove non-adherent cells. J Immunol Methods 187, 213-219 (1995).
46. Gyorffy B, et al. An online survival analysis tool to rapidly assess the effect of 22,277 genes on breast cancer prognosis using microarray data of 1,809 patients. Breast cancer research and treatment 123, 725-731 (2010).
47. Ketscher A, Ketterer S, Dollwet-Mack S, Reif U, Reinheckel T. Neuroectoderm-specific deletion of cathepsin D in mice models human inherited neuronal ceroid lipofuscinosis type 10. Biochimie 122, 219-226 (2016).

1. A method for treating cancer in a subject in need thereof comprising administering to the subject a therapeutically effective amount of an inhibitor of a SPARC fragment.

2. The method for treating cancer according to claim 1, wherein the SPARC fragment comprises or consists of a peptides selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, and SEQ ID NO:14.

3. The method for treating cancer according to claim 1, wherein the SPARC fragment is a C-terminal 9-kDa SPARC fragment.

4. The method for treating cancer according to claim 3, wherein the C-terminal 9-kDa SPARC fragment is a peptide selected from the group consisting of SEQ ID NO:5, SEQ ID NO:6, SEQ ID NO:7, SEQ ID NO:8, SEQ ID NO:9, SEQ ID NO:10, SEQ ID NO:11, SEQ ID NO:12, and SEQ ID NO:13.

5. The method for treating cancer according to claim 1, wherein the inhibitor of the SPARC fragment is an antibody, a peptide, a polypeptide, a small molecule or an aptamer.

6. The method for treating cancer according to claim 5, wherein the inhibitor of the SPARC fragment is an antibody.

7. The method for treating cancer according to claim 1, wherein the cancer is breast cancer.

8. The method for treating cancer according to claim 7, wherein the breast cancer is triple negative breast cancer.

9. The method for treating cancer according to claim 1, wherein the inhibitor of the SPARC fragment is administered in combination with a classical treatment of cancer.

10. A pharmaceutical composition comprising an inhibitor of a SPARC fragment.


SEQUENCE LISTING (41 sequences)

SEQ ID NO: 1 - protein, 257 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRA

SEQ ID NO: 2 - protein, 206 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRL

SEQ ID NO: 3 - protein, 128 aa, Homo sapiens
VTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 4 - protein, 125 aa, Homo sapiens
YERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 5 - protein, 74 aa, Homo sapiens
PVHWQFGQLDQHPIDGYLSHTELAPLRAPLIPMEHCTTRFFETCDLDNDKYIALDEWAGCFGIKQKDIDKDLVI

SEQ ID NO: 6 - protein, 71 aa, Homo sapiens
WQFGQLDQHPIDGYLSHTELAPLRAPLIPMEHCTTRFFETCDLDNDKYIALDEWAGCFGIKQKDIDKDLVI

SEQ ID NO: 7 - protein, 69 aa, Homo sapiens
FGQLDQHPIDGYLSHTELAPLRAPLIPMEHCTTRFFETCDLDNDKYIALDEWAGCFGIKQKDIDKDLVI

SEQ ID NO: 8 - protein, 68 aa, Homo sapiens
GQLDQHPIDGYLSHTELAPLRAPLIPMEHCTTRFFETCDLDNDKYIALDEWAGCFGIKQKDIDKDLVI

SEQ ID NO: 9 - protein, 67 aa, Homo sapiens
QLDQHPIDGYLSHTELAPLRAPLIPMEHCTTRFFETCDLDNDKYIALDEWAGCFGIKQKDIDKDLVI

SEQ ID NO: 10 - protein, 65 aa, Homo sapiens
DQHPIDGYLSHTELAPLRAPLIPMEHCTTRFFETCDLDNDKYIALDEWAGCFGIKQKDIDKDLVI

SEQ ID NO: 11 - protein, 58 aa, Homo sapiens
YLSHTELAPLRAPLIPMEHCTTRFFETCDLDNDKYIALDEWAGCFGIKQKDIDKDLVI

SEQ ID NO: 12 - protein, 57 aa, Homo sapiens
LSHTELAPLRAPLIPMEHCTTRFFETCDLDNDKYIALDEWAGCFGIKQKDIDKDLVI

SEQ ID NO: 13 - protein, 56 aa, Homo sapiens
SHTELAPLRAPLIPMEHCTTRFFETCDLDNDKYIALDEWAGCFGIKQKDIDKDLVI

SEQ ID NO: 14 - protein, 46 aa, Homo sapiens
PLIPMEHCTTRFFETCDLDNDKYIALDEWAGCFGIKQKDIDKDLVI

SEQ ID NO: 15 - protein, 11 aa, Homo sapiens
LDSELTEFPLR

SEQ ID NO: 16 - protein, 13 aa, Homo sapiens
DWLKNVLVTLYER

SEQ ID NO: 17 - protein, 16 aa, Homo sapiens
VTLYERDEDNNLLTEK

SEQ ID NO: 18 - protein, 13 aa, Homo sapiens
YERDEDNNLLTEK

SEQ ID NO: 19 - protein, 15 aa, Homo sapiens
YERDEDNNLLTEKQK

SEQ ID NO: 20 - protein, 12 aa, Homo sapiens
EAGDHPVELLAR

SEQ ID NO: 21 - protein, 27 aa, Homo sapiens
PVHWQFGQLDQHPIDGYLSHTELAPLR

SEQ ID NO: 22 - protein, 24 aa, Homo sapiens
WQFGQLDQHPIDGYLSHTELAPLR

SEQ ID NO: 23 - protein, 22 aa, Homo sapiens
FGQLDQHPIDGYLSHTELAPLR

SEQ ID NO: 24 - protein, 21 aa, Homo sapiens
GQLDQHPIDGYLSHTELAPLR

SEQ ID NO: 25 - protein, 20 aa, Homo sapiens
QLDQHPIDGYLSHTELAPLR

SEQ ID NO: 26 - protein, 18 aa, Homo sapiens
DQHPIDGYLSHTELAPLR

SEQ ID NO: 27 - protein, 11 aa, Homo sapiens
YLSHTELAPLR

SEQ ID NO: 28 - protein, 10 aa, Homo sapiens
LSHTELAPLR

SEQ ID NO: 29 - protein, 9 aa, Homo sapiens
SHTELAPLR

SEQ ID NO: 30 - protein, 11 aa, Homo sapiens
PLIPMEHCTTR

SEQ ID NO: 31 - protein, 10 aa, Homo sapiens
APQQEALPDE

SEQ ID NO: 32 - protein, 12 aa, Homo sapiens
APQQEALPDETE

SEQ ID NO: 33 - protein, 15 aa, Homo sapiens
APQQEALPDETEVVE

SEQ ID NO: 34 - protein, 16 aa, Homo sapiens
APQQEALPDETEVVEE

SEQ ID NO: 35 - protein, 20 aa, Homo sapiens
APQQEALPDETEVVEETVAE

SEQ ID NO: 36 - protein, 15 aa, Homo sapiens
VTLYERDEDNNLLTE

SEQ ID NO: 37 - protein, 12 aa, Homo sapiens
YERDEDNNLLTE

SEQ ID NO: 38 - protein, 17 aa, Homo sapiens
FGQLDQHPIDGYLSHTE

SEQ ID NO: 39 - protein, 16 aa, Homo sapiens
GQLDQHPIDGYLSHTE

SEQ ID NO: 40 - protein, 13 aa, Homo sapiens
DQHPIDGYLSHTE

SEQ ID NO: 41 - protein, 150 aa, Homo sapiens
PCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI